SEQUENCE LISTING (24 sequences)

SEQ ID NO: 1 - DNA, 50 bp, Exon 10 of Human Cystic Fibrosis Gene
tggcaccattaaagaaaatatcatctttggtgtttcctatgatgaatata

SEQ ID NO: 2 - DNA, 50 bp, Exon 10 of Human Cystic Fibrosis Gene
tggcaccattaaagaaaatatcgtctttggtgtttcctatgatgaatata

SEQ ID NO: 3 - DNA, 50 bp, Exon 10 of Human Cystic Fibrosis Gene
tggcaccattaaagaaaatatactctttggtgtttcctatgatgaatata

SEQ ID NO: 4 - DNA, 50 bp, Exon 10 of Human Cystic Fibrosis Gene
tggcaccattaaagaaaatatacgctttggtgtttcctatgatgaatata

SEQ ID NO: 5 - DNA, 50 bp, Exon 10 of Human Cystic Fibrosis Gene
gagcaccatgacagacactgtcatctctggtgtgtcctacgatgactctg

SEQ ID NO: 6 - DNA, 50 bp, Exon 10 of Human Cystic Fibrosis Gene
gagcaccatgacagacactgtcgtctctggtgtgtcctacgatgactctg

SEQ ID NO: 7 - DNA, 50 bp, Exon 10 of Human Cystic Fibrosis Gene
gagcaccatgacagacactgtcatctttggtgtgtcctacgatgactctg

SEQ ID NO: 8 - DNA, 50 bp, Exon 10 of Human Cystic Fibrosis Gene
gagcaccatgacagacactgtactctctggtgtgtcctacgatgactctg

SEQ ID NO: 9 - DNA, 47 bp, Exon 10 of Human Cystic Fibrosis Gene
tggcaccattaaagaaaatatcattggtgtttcctatgatgaatata

SEQ ID NO: 10 - DNA, 50 bp, Exon 10 of Human Cystic Fibrosis Gene
gagcaccatgacagacactgtcttctctggtgtgtcctacgatgactctg

SEQ ID NO: 11 - DNA, 50 bp, Exon 10 of Human Cystic Fibrosis Gene
gagcaccatgacagacactgtcatccctggtgtgtcctacgatgactctg

SEQ ID NO: 12 - DNA, 15 bp, Exon 10 of Human Cystic Fibrosis Gene
  misc_feature ()..(): 5′ end begins with H, 3′ end ends with lysine-CONH2
caccaaagatgatat

SEQ ID NO: 13 - DNA, 15 bp, Exon 10 of Human Cystic Fibrosis Gene
  misc_feature ()..(): 5′ end begins with H, 3′ end ends with lysine-CONH2
tatagtagaaaccac

SEQ ID NO: 14 - DNA, 15 bp, Exon 10 of Human Cystic Fibrosis Gene
caccagagatgacag

SEQ ID NO: 15 - DNA, 15 bp, Exon 10 of Human Cystic Fibrosis Gene
gacagtagagaccac

SEQ ID NO: 16 - DNA, 15 bp, Exon 10 of Human Cystic Fibrosis Gene
  misc_feature ()..(): fluorescein moiety attached at the 5′ position
caccagagatgacag

SEQ ID NO: 17 - DNA, 15 bp, Exon 10 of Human Cystic Fibrosis Gene
  misc_feature ()..(): fluorescein moiety attached at 5′ end
gacagtagagaccac

SEQ ID NO: 18 - DNA, 15 bp, Exon 10 of Human Cystic Fibrosis Gene
  misc_feature ()..(): fluorescein moiety attached at 5′ end
caccaaagatgatat

SEQ ID NO: 19 - DNA, 15 bp, Exon 10 of Human Cystic Fibrosis Gene
caccaaagatgatat

SEQ ID NO: 20 - DNA, 15 bp, Exon 10 of Human Cystic Fibrosis Gene
cacgaaagatgatat

SEQ ID NO: 21 - DNA, 15 bp, Exon 10 of Human Cystic Fibrosis Gene
caccaaacatgatat

SEQ ID NO: 22 - DNA, 15 bp, Exon 10 of Human Cystic Fibrosis Gene
  misc_feature ()..(): fluorescein moiety attached at 5′ end
ctgtcatctctggtg

SEQ ID NO: 23 - DNA, 15 bp, Exon 10 of Human Cystic Fibrosis Gene
ctgtcatctctggtg

SEQ ID NO: 24 - DNA, 15 bp, Exon 10 of Human Cystic Fibrosis Gene
gacagtagagaccac

(19) United States
(12) Patent Application Publication
(10) Pub. No.: US 2003/0181412 A1
(43) Pub. Date: Sep. 25, 2003

(54) Method for modifying transcription and/or translation in an organism for therapeutic, prophylactic and/or analytic uses

(75) Inventor: Glen H. Erikson, Providenciales (TC)

Correspondence Address: CAESAR, RIVISE, BERNSTEIN,, COHEN & POKOTILOW, LTD., 12TH FLOOR, SEVEN PENN CENTER, 1635 MARKET STREET, PHILADELPHIA, PA, 19103-2212, US

(73) Assignee: Ingeneus Corporation, Bridgetown (BB)

(21) Appl. No.: 10/438,151

(22) Filed: May 14, 2003

Related U.S. Application Data
(63) Continuation-in-part of application No. 09/909,496, filed on Jul. 20, 2001.
Application No. 09/909,496 is a continuation-in-part of application No. 09/613,263, filed on Jul. 10, 2000, now Pat. No. 6,420,115.
Application No. 09/909,496 is a continuation-in-part of application No. 09/468,679, filed on Dec. 21, 1999, now Pat. No. 6,403,313.
(63) Continuation-in-part of application No. 09/664,827, filed on Sep. 19, 2000.

(51) Int. Cl. A61K 48/00; C12Q 1/68

(52) U.S. Cl. 514/44; 435/6

(57) ABSTRACT

A method for modifying transcription and/or translation in an organism (e.g., for preventing or treating a disorder in the organism), includes administering to the organism a composition containing a probe containing a heteropolymeric probe sequence of nucleic acids or nucleic acid analogues; and binding the probe to a target to modify transcription and/or translation in the organism, wherein the target is in the organism and contains a heteropolymeric target sequence of nucleic acids, and wherein the heteropolymeric probe sequence is bonded to the heteropolymeric target sequence to form a complex by Watson-Crick complementary base interaction or by homologous base interaction, provided that when the complex is a duplex and the heteropolymeric probe sequence is antiparallel to the heteropolymeric target sequence, the heteropolymeric probe sequence is bonded to the heteropolymeric target sequence by homologous base interaction, and provided that when the complex is a triplex, the complex is preferably free of RecA protein.

### BACKGROUND OF THE INVENTION

1. FIELD OF INVENTION

The invention relates to a method for modifying gene expression for therapeutic and/or prophylactic purposes, and more particularly to such a method wherein duplex, triplex and/or quadruplex complexes are formed by specific binding between single-stranded or double-stranded nucleobase-containing probes and single-stranded or double-stranded nucleobase-containing target sequences.

2. DESCRIPTION OF RELATED ART

The Watson-Crick model of nucleic acids has been the accepted standard in molecular biology for nearly fifty years.

Although antiparallel nucleic acid duplexes first suggested by Watson and Crick are the most widely studied type of multiple-strand nucleic acid structures, it has been discovered that nucleic acids also form triplex structures and quadruplex structures under certain conditions.

Until recently, for instance, binding among three nucleic acid strands to form a triplex was widely believed to be confined to very limited species of nucleic acids (e.g., polypurine or polypyrimidine sequences). See, e.g., Floris et al., “Effect of cations, on purine-purine-pyrimidine triple helix formation in mixed-valence salt solutions,” 260 Eur. J. Biochem. 801-809 (1999). Moreover, canonical triplex binding or hybridization was thought to be based on Hooqgsteen binding between limited varieties of nucleobase sequences, rather than Watson-Crick base recognition. See, e.g., Floris et al. and U.S. Pat. No. 5,874,555 to Dervan et al. However, a related group of inventors has recently disclosed in several patents that specifically bound mixed base sequence triplex nucleic acids based on Watson-Crick base pairing recognition can be created and used as the basis for a highly accurate and sensitive assay for specific binding. See U.S. Pat. No. 6,420,115 to Erikson et al. and U.S. Pat. No. 6,403,313 to Daksis et al. These prior developments are based upon a model wherein stably paired bases in a conventional nucleic acid duplex can be rendered specifically identifiable by bases in a third strand.

Zhurkin et al., 239 J. Mol. Biol. 181 (1994) discloses the possibility of parallel DNA triplexes; however, these triplexes are said to be only created by the third strand binding in the major groove of the duplex in the presence of recombination proteins, such as RecA protein.

As has been the case with triplex nucleic acids, the conventional wisdom regarding quadruplex nucleic acids has been that such peculiar structures only exist under relatively extreme conditions and only for a narrow class of nucleic acids. In particular, Sen et al. (Nature 334:364-366 (1988)) disclosed that guanine-rich oligonucleotides can spontaneously self-assemble into four-stranded helices in vitro. Sen et al. (Biochemistry 31:65-70 (1992)) disclosed that these four-stranded complexes can further associate into superstructures composed of 8, 12, or 16 oligomers.

Marsh et al. (Biochemistry 33:10718-10724 (1994), and Nucleic Acids Research 23:696-700 (1995)) disclosed that some guanine-rich oligonucleotides can also assemble in an offset, parallel alignment, forming long “G-wires”. These higher-order structures are stabilized by G-quartets that consist of four guanosine residues arranged in a plane and held together through Hoogsteen base pairings. According to Sen et al. (Biochemistry 31:65-70 (1992)), at least three contiguous guanines within the oligomer are critical for the formation of these higher order structures.

It has been suggested that four-stranded DNAs play a role in a variety of biological processes, such as inhibition of HIV-1 integrase (Mazumder et al., Biochemistry 35:13762-13771 (1996)), formation of synapsis during meiosis (Sen et al., Nature 334:364-366 (1988)), and telomere maintenance (Williamson et al., Cell 59:871-880 (1989)); Baran et al., Nucleic Acids Research 25:297-303 (1997)). It has been further suggested that controlling the production of guanine-rich quadruplexes might be the key to controlling such biological processes. For example, U.S. Pat. No. 6,017,709 to Hardin et al. suggests that telomerase activity might be controlled through drugs that inhibit the formation of guanine quartets.

U.S. Pat. No. 5,888,739 to Pitner et al. discloses that G-quartet based quadruplexes can be employed in an assay for detecting nucleic acids.

U.S. Pat. No. 5,912,332 to Agrawal et al. discloses a method for the purification of synthetic oligonucleotides, wherein the synthetic oligonucleotides hybridize specifically with a desired, full-length oligonucleotide and concomitantly form a multimer aggregate, such as quadruplex DNA. The multimer aggregate containing the oligonucleotide to be purified is then isolated using size-exclusion techniques.

U.S. Pat. No. 6,432,638 to Dervan et al. discloses homopyrimidinepolydeoxyribonucleotide probes with at least one detectable marker, chemotherapeutic agent or a DNA-cleaving moiety attached to at least one predetermined position. See also U.S. Pat. No. 6,403,302 to Dervan et al. The probes are said to be capable of binding the corresponding homopyrimidine-homopurine tracts within large double-stranded nucleic acids by triple-helix formation at a predetermined site, and can be used for gene therapy.

U.S. Pat. No. 5,650,316 to Aggarwal et al. discloses antisense therapy using triplex-forming homopolymeric oligonucleotides. U.S. Pat. No. 6,395,474 to Buchardt et al. discloses compositions comprising peptide nucleic acids, and their use for sequence-specific recognition and inactivation of dsDNA targets through a strand-invasion mechanism. Strand-invasion is said to differ significantly from “triple helix recognition” in that the latter is largely limited to recognition of homopurine-homopyrimidine sequences at non-physiological conditions.

U.S. Pat. No. 6,506,559 to Fire et al. discloses the use of dsRNA to inhibit gene expression in a target gene in a living cell. It is disclosed that the precise mechanism of action is unclear; however, the patent discloses that “extreme environmental and sequence constraints on triple-helix formation make it unlikely that dsRNA-mediated inhibition . . . is mediated by a triple-strand structure.”

In view of the foregoing perceived constraints and prohibitions, it is desired to provide therapeutic, prophylactic and analytic complexes and methods comprising parallel or antiparallel, homologous or complementary nucleic acids or analogues thereof based upon specific nucleic acid binding capabilities demonstrated or elucidated herein.

All references cited herein are incorporated herein by reference in their entireties.

### BRIEF SUMMARY OF THE INVENTION

Accordingly, the invention provides a method for modifying transcription and/or translation in an organism, said method comprising:

administering to the organism a composition comprising a probe containing a heteropolymeric probe sequence of nucleic acids or nucleic acid analogues; and

binding the probe to a target to modify transcription and/or translation in the organism, wherein the target is in the organism and contains a heteropolymeric target sequence of nucleic acids,

wherein the heteropolymeric probe sequence is bonded to the heteropolymeric target sequence to form a complex by Watson-Crick complementary base interaction or by homologous base interaction, provided that when the complex is a duplex and the heteropolymeric probe sequence is antiparallel to the heteropolymeric target sequence, the heteropolymeric probe sequence is bonded to the heteropolymeric target sequence by homologous base interaction, and provided that when the complex is a triplex, the complex is preferably free of RecA protein.

Also provided is a method for preventing or treating a disorder in an organism, said method comprising:

administering to the organism a composition comprising a probe containing a heteropolymeric probe sequence of nucleic acids or nucleic acid analogues; and

binding the probe to a target to prevent or treat the disorder in the organism, wherein the target is in the organism and contains a heteropolymeric target sequence of nucleic acids,

wherein the heteropolymeric probe sequence is bonded to the heteropolymeric target sequence to form a complex by Watson-Crick complementary base interaction or by homologous base interaction, provided that when the complex is a duplex and the heteropolymeric probe sequence is antiparallel to the heteropolymeric target sequence, the heteropolymeric probe sequence is bonded to the heteropolymeric target sequence by homologous base interaction, and provided that when the complex is a triplex, the complex is preferably free of RecA protein.

### BRIEF DESCRIPTION OF SEVERAL VIEWS OF THE DRAWINGS

The invention will be described in conjunction with the following drawings in which like reference numerals designate like elements and wherein:

FIGS. 1A, 1B, 1C, 2A, 2B, 3A, 3B, 4, 5A, 5B, 6, 7, 8, 9, 10A, 10B, 11A, 11B, 12A, 12B, 13A and 13B are composite graphs of fluorescent intensity plotted as a function of wavelength for each sample analyzed; and

FIGS. 14A, 14B and 14C are composite graphs of fluorescent intensity plotted as a function of time for each sample analyzed.

### DETAILED DESCRIPTION OF THE INVENTION

The invention flows from our elucidation of the specific binding properties of heteropolymeric nucleic acid strands. We have previously disclosed the specific binding of a heteropolymeric strand to duplex nucleic acid and the specific binding of duplex nucleic acid to other duplex nucleic acid. We have also disclosed that heteropolymeric nucleic acids (and/or their analogues) can specifically bind to each other by homologous base bonding as well as by Watson-Crick base interaction, and that base bonding is not limited to strands having antiparallel directionality relative to each other. Thus, heteropolymeric nucleic acids (and/or their analogues) can specifically bind to each other with parallel or antiparallel directionality, wherein the bases bond by homologous base bonding and/or Watson-Crick base bonding rules. All of the foregoing is readily reproducible having been detected in vitro using readily available instruments and reagents used under mild and permissive conditions.

The invention is more than merely the disclosure of unorthodox binding properties of nucleic acids. The present invention focuses on gene therapy methods being therapeutic methods, prophylactic methods and analytic methods. Formation of the complexes provided will tend to prohibit or inhibit transcription or translation in the organism for a therapeutic, prophylactic or analytic purpose.

The invention encompasses the use and/or formation of novel duplex, triplex and quadruplex complexes of nucleic acids (and/or analogues thereof).

Nucleic acid strands have inherent directionality. The conventional wisdom holds that strands of opposite directionality, i.e., which are antiparallel in their orientation to one another, can form a duplex through Watson-Crick complementary binding of their respective bases.

Certain duplexes according to the invention, on the other hand, comprise two strands of nucleic acid (and/or nucleic acid analogues) hybridized in parallel relation to one another, wherein specific binding is either through homologous base pairing or Watson-Crick base pairing. Conventional wisdom holds that such duplexes do not exist, or at least would be extremely unstable due to, e.g., backbone irregularities necessitated by the conformational requirements of parallel base bonding. Even more surprising is our discovery that under appropriate hybridization conditions, homologous bonding demonstrates specificity and stability rivaling that of Watson-Crick complementary antiparallel duplex.

The invention also encompasses duplexes containing two strands of nucleic acid (and/or nucleic acid analogues) hybridized in antiparallel relation to one another, wherein specific binding is through homologous base pairing.

As used herein, the terms “Watson-Crick base pairing”, “complementary base pairing” and the like are intended to define specific association between opposing or adjacent pairs of nucleic acid and/or nucleic acid analogue strands via matched bases (e.g., A:T; G:C and/or A:U). In the context of non-canonical complexes described herein, including parallel duplexes, parallel and antiparallel triplexes, and parallel and antiparallel quadruplexes, terms like “Watson-Crick base bonding” and “complementary base bonding” are intended to denote bonding between A and T, A and U and/or G and C, but not necessarily in the edgewise, planar conformation first described by Watson and Crick. In addition to the conventional binding motif first proposed by Watson and Crick (the “W-C motif”), and conformational variants thereof encompassed by the foregoing definition of Watson-Crick base bonding, the present invention encompasses complexes formed by homologous base bonding. In homologous base bonding, bases bond specifically with identical bases rather than complementary bases. Thus, in the “homologous motif”, homologous base pairs include A:A, G:G, C:C, T:T, U:U, and T:U.

The binding by the bases of nucleic acid strands is affected or conditioned by a number of factors, particularly the binding potential of the strands pursuant to either the W-C motif or homologous motif, and ionic conditions (e.g., salt concentration and/or type). Salty conditions tend to favor the formation of Watson-Crick bonding over homologous bonding. Homologous motif quadruplexes are favored over W-C motif quadruplexes under identical buffer conditions probably because the localized environment can become relatively low-salt, based on the presence of the charged backbones of the two duplex nucleic acids.

Each strand in a complex of the invention can comprise any sequence of nucleobases and/or nucleobase analogues, provided the nucleobases are related to the nucleobases to which they are to specifically bind by either the W-C motif or the homologous motif. Contrary to certain teachings of the prior art, the target and probe need not be homopolymeric to achieve binding, even in the case of triplex or quadruplex formation. Thus, in certain embodiments, the probe nucleobases are arranged in a heteropolymeric probe sequence of interspersed purines and pyrimidines, and the target nucleobases are arranged in a target sequence at least partially complementary or partially homologous to the probe sequence. For example, the probe sequence can contain 25% to 75% purine bases and 75% to 25% pyrimidine bases in any order. Complexes of the invention can form from heteropolymeric sequences, which as defined herein, means sequences containing at least one purine nucleobase or purine analogue and at least one pyrimidine nucleobase or pyrimidine analogue in at least their hybridizing segments. Heteropolymeric sequences preferably lack homopolymeric fragments greater than 5 bases long. Other nucleobases are also suitable for use in the invention, such as, e.g., synthetic analogues of naturally occurring bases, which have specific Watson-Crick and/or homologous binding affinities to other bases.

In addition to duplexes, complexes of the invention also include triplexes and quadruplexes, wherein opposing heteropolymeric strands are linked by Watson-Crick complementary bases or by homologous bases, and the relative directionality of the bound sequences is parallel or antiparallel to one another.

A probe strand can specifically bind in the major or minor groove of a double-stranded target. Further, the bases of a single-stranded probe can interact specifically with bases on one or both strands of a double-stranded target. Similarly, the bases of each strand of a double-stranded probe can interact specifically with bases on one or both strands of a double-stranded target in quadruplex complexes of the invention.

In certain triplex and quadruplex embodiments, each nucleobase binds to one or two other nucleobases. Thus, in addition to the traditional duplex Watson-Crick base pairs and the duplex homologous base pairs described above, such embodiments include the following Watson-Crick base binding triplets: A:T:A, T:A:T, U:A:T, T:A:U, A:U:A, U:A:U, G:C:G and/or C:G:C (including C⁺:G:C, and/or any other ionized species of base), and/or the following homologous base triplets: A:A:T, T:T:A, U:U:A, T:U:A, A:A:U, U:T:A, G:G:C and/or C:C:G (including C:C⁺:G, and/or any other ionized species of base).

Thus, in certain quadruplex embodiments wherein the probe is defined as a duplex of a first and a second strand and the target is defined as a duplex of a third and a fourth strand, it is believed that the bases of the first and third strands also bind to each other, in addition to: (a) the binding between opposing bases of the first and second strands; (b) the binding between opposing bases of the third and fourth strands; and (c) the binding between opposing bases of the second and fourth strands.

In certain embodiments of the triplex and quadruplex structures of the invention, no binding sequence of bases is contiguous with another binding sequence of bases. That is, there are at least three separate strands. Although folded conformations and the like (e.g., hairpin turns, etc.) are within the scope of the invention (particularly but not limited to RNA interference embodiments, where hairpin design has been found to be advantageous for causing interference based on conventional Watson-Crick duplex binding of RNA targets—see Hemann et al., “An epi-allelic series of p53 hypomorphs created by stable RNAi produces distinct tumor phenotypes in vivo.” Nat. Genet. 2003 Mar; 33(3):396-400.), folded portions of a single strand do not make the strand count more than once toward the minimum of three separate strands.

Complexes of the invention preferably do not rely on Hoogsteen bonding (including reverse Hoogsteen bonding) or G—G quartets for maintenance of the complex structure, although Hoogsteen bonding (including reverse Hoogsteen bonding) and/or G—G quartets may be present. That is, complexes of the invention are preferably substantially free of Hoogsteen bonding (including reverse Hoogsteen bonding), and substantially free of G—G quartets.

Each strand of the complex independently comprises a nucleic acid having a deoxyribose phosphate or ribose phosphate backbone (e.g., DNA, RNA, mRNA, hnRNA, rRNA, tRNA or cDNA) or a nucleic acid analogue. Preferred nucleic acid analogues contain an uncharged or partially charged backbone (i.e., a backbone having a charge that is not as negative as a native DNA backbone), and include, e.g., PNA and LNA. Certain embodiments are free of PNA. For increased stability, probes can be provided in a phosphotriester form, to inhibit degradation during use.

Unlike certain complexes particularly associated with PNA, triplexes of the invention do not depend upon a strand invasion mechanism.

At least a portion of the complex is isolated, purified, artificial or synthetic.

In embodiments, a portion of the complex is a PCR amplified product.

The complexes of the invention can be present in solution, on a solid support, in vitro, in vivo or in silico. The solid support can be electrically conductive (e.g., an electrode) or non-conductive. In addition, the complexes can be optically mapped or sequenced after being elongated, as taught in U.S. Pat. Nos. 6,147,198 and 5,720,928 to Schwartz.

Specific binding between complementary bases occurs under a wide variety of conditions having variations in temperature, salt concentration, electrostatic strength, and buffer composition. Examples of these conditions and methods for applying them are known in the art. Our copending U.S. patent application Ser. No. 09/885,731, filed Jun. 20, 2001, discloses conditions particularly suited for use in this invention.

Unlike many Hoogsteen-type complexes, which are unstable or non-existent at pH levels above about 7.6, the complexes of the invention are stable over a wide range of pH levels, preferably from about pH 5 to about pH 9.

The invention can be used to prevent and/or treat conditions associated with infection by an organism or virus. Likely therapeutic and prophylactic targets include, but are not limited to, herpes simplex virus (HSV), human papillomavirus (HPV), human immunodeficiency virus (HIV), candidia albicans, influenza virus, cytomegalovirus (CMV), Epstein Barr Virus (EBV), intracellular adhesion molecules (ICAM), 5-lipoxygenase (5-LO), phospholipase A₂ (PLA₂), protein kinase C (PKC), and RAS oncogene. Potential applications of such targeting include treatments for ocular, labial, genital, and systemic herpes simplex I and II infections; genital warts; cervical cancer; common warts; Kaposi's sarcoma; AIDS; skin and systemic fungal infections; flu; pneumonia; retinitis and pneumonitis in immunosuppressed patients; mononucleosis; ocular, skin and systemic inflammation; cardiovascular disease; cancer; asthma; psoriasis; cardiovascular collapse; cardiac infarction; gastrointestinal disease; kidney disease; rheumatoid arthritis; osteoarthritis; acute pancreatitis; septic shock; and Crohn's disease.

The probe may be directly introduced into the cell (i.e., intracellularly); or introduced extracellularly into a cavity, interstitial space, into the circulation of an organism, introduced orally, or may be introduced by bathing an organism in a solution containing the probe. Methods for oral introduction include direct mixing of the probe with food of the organism, as well as engineered approaches in which a species that is used as food is engineered to express the probe, then fed to the organism to be affected. For example, the probe may be sprayed onto a plant or a plant may be genetically engineered to express the probe in an amount sufficient to kill some or all of a pathogen known to infect the plant. Physical methods of introducing nucleic acids, for example, injection directly into the cell or extracellular injection into the organism, may also be used. Vascular or extravascular circulation, the blood or lymph system, the phlegm, the roots, and the cerebrospinal fluid are sites where the probe may be introduced. A transgenic organism that expresses the probe from a recombinant construct may be produced by introducing the construct into a zygote, an embryonic stem cell, or another multipotent cell derived from the appropriate organism.

It is also possible to introduce the probe into an organism containing the target by introducing the probe into a bacterium, which is then internalized by the organism.

Physical methods of introducing nucleic acids include injection of a solution containing the probe, bombardment by particles covered by the probe, soaking the cell or organism in a solution of the probe, or electroporation of cell membranes in the presence of the probe. A viral construct packaged into a viral particle would accomplish both efficient introduction of an expression construct into the cell and transcription of RNA encoded by the expression construct. Other methods known in the art for introducing nucleic acids to cells may be used, such as lipid-mediated carrier transport, bacterial transport, chemical-mediated transport, such as calcium phosphate, and the like. Thus the probe may be introduced along with components that perform one or more of the following activities: enhance nucleic acid uptake by the cell, promote annealing of the duplex, triplex or quadruplex strands, stabilize the annealed strands, or otherwise increase inhibition of the target gene.

For therapeutic, prophylactic or analytic treatment, the probes of the invention can be formulated in a pharmaceutical composition, which can include carriers, thickeners, diluents, buffers, preservatives, surface active agents and the like. Pharmaceutical compositions may also include one or more active ingredients, such as antimicrobial agents, anti-inflammatory agents, anesthetics, and the like, in addition to the probe.

The pharmaceutical composition may be administered in a number of ways depending on whether local or systemic treatment is desired, and on the area to be treated. Administration may be done topically (including ophthalmically, vaginally, rectally, intranasally), orally, by inhalation, or parenterally, for example by intravenous drip or subcutaneous, intraperitoneal or intramuscular injection.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable. Coated condoms may also be useful.

Compositions for oral administration include powders or granules, suspensions or solutions in water or non-aqueous media, capsules, sachets, or tablets. Thickeners, flavorings, diluents, emulsifiers, dispersing aids or binders may be desirable.

Formulations for parenteral administration may include sterile aqueous solutions, which may also contain buffers, diluents and other suitable additives.

Dosing is dependent on severity and responsiveness of the condition to be treated, or the expression or phenotype to be affected, but will normally be one or more doses per day, with course of treatment lasting from several days to several months or until a cure is effected or a diminution of disease state is achieved. Prophylactic applications may require periodic, prolonged or permanent dosing. Persons of ordinary skill can easily determine optimum dosages, dosing methodologies and repetition rates.

Treatments of this type can be practiced on a variety of organisms ranging from unicellular prokaryotic and eukaryotic organisms to multicellular eukaryotic organisms. Any organism that utilizes DNA-RNA transcription or RNA-protein translation as a fundamental part of its hereditary, metabolic or cellular control is susceptible to therapeutic and/or prophylactic treatment in accordance with the invention. Seemingly diverse organisms such as bacteria, yeast, protozoa, algae, all plants and all higher animal forms, including warm-blooded animals, can be treated. Further, each cell of multicellular eukaryotes can be treated since they include both DNA-RNA transcription and RNA-protein translation as integral parts of their cellular activity. Furthermore, many of the organelles (e.g., mitochondria and chloroplasts) of eukaryotic cells also include transcription and translation mechanisms. Thus, single cells, cellular populations or organelles can also be included within the definition of organisms that can be treated with therapeutic or diagnostic oligonucleotides. As used herein, “therapeutics” is meant to include the eradication of a disease state, by killing an organism or by control of erratic or harmful cellular growth or expression.

As used herein, a “pharmaceutically acceptable” component is one that is suitable for use with humans and/or animals without undue adverse side effects (such as toxicity, irritation, and allergic response) commensurate with a reasonable benefit/risk ratio.

As used herein, the term “safe and effective amount” refers to the quantity of a component, which is sufficient to yield a desired therapeutic response without undue adverse side effects (such as toxicity, irritation, or allergic response) commensurate with a reasonable benefit/risk ratio when used in the manner of this invention. The specific “safe and effective amount” will, obviously, vary with such factors as the particular condition being treated, the physical condition of the patient, the type of mammal being treated, the duration of the treatment, the nature of concurrent therapy (if any), and the specific formulations employed and the structure of the compounds or its derivatives.

As used herein, a “pharmaceutical carrier” is a pharmaceutically acceptable solvent, suspending agent or vehicle for delivering the probe to the animal or human. The carrier may be liquid or solid and is selected with the planned manner of administration in mind.

Complexes of the invention can be formed under conventional hybridization conditions, under triplex hybridization conditions, under quadruplex hybridization conditions or under conditions of in situ hybridization. It is preferred that complexes be formed at a temperature of about 2° C. to about 55° C. for about two hours or less. In certain embodiments, the incubation time is preferably less than five minutes, even at room temperature. Longer reaction times may not be required, but incubation for up to 24 hours in most cases does not adversely affect the complexes. The fast binding times of the complexes of the invention contrast with the much longer binding times necessary for Hoogsteen bound complexes. Accordingly, complexes of the invention can be formed under physiological conditions present in a variety of organisms.

A promoter can be used in combination with the probe. The promoter is preferably an intercalating agent or a cation, as disclosed in U.S. Pat. No. 6,420,115 to Erikson et al. The intercalators are optionally fluorescent. The intercalating agent can be, e.g., a fluorophore, such as a member selected from the group consisting of YOYO-1, TOTO-1, YOYO-3, TOTO-3, POPO-1, BOBO-1, POPO-3, BOBO-3, LOLO-1, JOJO-1, cyanine dimers, YO-PRO-1, TO-PRO-1, YO-PRO-3, TO-PRO-3, TO-PRO-5, PO-PRO-1, BO-PRO-l, PO-PRO-3, BO-PRO-3, LO-PRO-1, JO-PRO-1, cyanine monomers, ethidium bromide, ethidium homodimer-1, ethidium homodimer-2, ethidium derivatives, acridine, acridine orange, acridine derivatives, ethidium-acridine heterodimer, ethidium monoazide, propidium iodide, SYTO dyes, SYBR Green 1, SYBR dyes, Pico Green, SYTOX dyes and 7-aminoactinomycin D.

Suitable cations include, e.g., monovalent cations, such as Na⁺, and other alkali metal ions; divalent cations, such as alkaline earth metal ions (e.g., Mg⁺² and Ca⁺²) and divalent transition metal ions (e.g., Mn⁺², Ni⁺², Cd⁺², Co⁺² and Zn⁺²); and cations having a positive charge of at least three, such as Co(NH₃)₆⁺³, trivalent spermidine and tetravalent spermine.

The amount of cation added to the medium in which the complex forms depends on a number of factors, including the nature of the cation, the concentration of probe, the concentration of target, the presence of additional cations and the base content of the probe and target. The preferred cation concentrations and mixtures can routinely be discovered experimentally. For triplexes, it is preferred to add cation(s) to the medium in the following amounts: (a) 10 mM-30 mM Mn⁺²; (b) 10 mM-20 mM Mg⁺²; (c) 20 mM Ni⁺²; or (d) 1 mM-30 mM of each of Mn⁺² and Mg⁺². For quadruplexes, it is preferred to add cation(s) to the medium in the following amounts: (a) 10 mM-45 mM Mn 2; (b) 10 mM-45 mM Mg⁺²; or (c) 10 mM-40 mM of each of Mn⁺² and Mg⁺². Where it is impractical to add cations to the medium (as it may be when the complex is formed in vivo), the localized cation concentration may be adjusted by binding the cation to the probe. In some of these embodiments, it will be desirable to employ a cation release agent to free the cations from their bonds as desired. Alternatively, local cation concentration may be adjusted by focusing the dosing in a particular tissue or organ where targets are highly concentrated and/or from which targets originate.

Although not required, other promoters include, e.g., single stranded binding proteins such as Rec A protein, T4 gene 32 protein, E. coli single stranded binding protein, major or minor nucleic acid groove binders, viologen and additional intercalating substances such as actinomycin D, psoralen, and anqelicin. Such facilitating reagents may prove useful in extreme operating conditions, for example, under abnormal pH levels or extremely high temperatures. Certain methods for providing complexes of the invention are conducted in the absence of protein promoters, such as Rec A and/or other recombination proteins.

The promoter is preferably covalently bound to the probe. When the promoter is covalently bound to the probe, it is preferably bound to the probe at either end.

In certain embodiments, it is preferred that the probe comprise active agents, including but not limited to, chemotherapeutic agents and other drugs, nucleic acid cleaving agents (e.g., metal chelators containing iron, and other examples disclosed in U.S. Pat. No. 6,432,638 to Dervan et al.), nucleases, polymerases, transcription factors, crosslinking agents, intercalators, minor groove binders, photoactive agents, labels, duplex binding agents, triplex binding agents, quadruplex binding agents, multimeric binding agents, proteins, peptides, and/or recombinases. The probe can also contain spacers, linkers, anchoring agents (adapted to bind to other anchoring agents) and/or blocking agents. The blocking agents are preferably located on at least one terminus of the probe, such that the probe is resistant to digestion by the organism.

In certain embodiments, at least one base of the probe can be a radical cation.

In certain embodiments, the probe can be provided as a product of transcription, expression or digestion in a cell or an organism. The probe can comprise a vector, a transfer vehicle, a transfection vehicle or a cell-uptake component.

In certain embodiments, at least one free base, nucleotide, nucleoside, cationic polypeptide, monovalent cation or transition metal cation bonded to at least one of the probe and the target. See copending U.S. patent application Ser. No. 10/346,752, filed Jan. 17, 2003.

Suitable targets are preferably 8 to 3.3×10⁹ base pairs long or longer, and can be single or double-stranded. Targets are preferably genomic material of the organism being treated or of another organism (e.g., viral or bacterial pathogens) within the organism being treated by the method of the invention. The target can comprise a promoter sequence, a coding sequence, a promoter sequence and an adjacent coding sequence, a non-coding sequence or a repetitive sequence. The target can comprise DNA (e.g., genomic DNA, rDNA and mDNA) or RNA (e.g., mRNA, hnRNA, mtRNA, rRNA, tRNA or snRNA).

In certain embodiments, the target comprises nucleic acids that are methylated, telomeric or in an A, B or Z conformation.

A target gene can be bound to a probe of the invention to hinder expression of the gene or to alter phenotype of the organism. Transcription can be partially or completely silenced. The target gene may be a gene derived from the cell, an endogenous gene, a transgene, or a gene of a pathogen, which is present in the cell after infection thereof. Depending on the particular target gene and the dose of probe delivered, the procedure may provide partial or complete loss of function for the target gene. Cell apoptosis or necrosis is also possible.

Inhibition of gene expression refers to the absence (or observable decrease) in the level of protein and/or mRNA product from a target gene. Specificity refers to the ability to inhibit the target gene without manifest effects on other genes of the cell. The consequences of inhibition can be confirmed by examination of the outward properties of the cell or organism (as presented below in the examples) or by biochemical techniques such as RNA solution hybridization, nuclease protection, fluorescent in situ hybridization, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). Gene expression is conveniently assayed by use of a reporter or drug resistance gene whose protein product is easily assayed. Such reporter genes include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentamycin, hygromycin, kanamycin, lincomycin, methotrexate, phosphinothricin, puromycin, and tetracyclin.

Depending on the assay, quantitation of the amount of gene expression allows one to determine a degree of inhibition which is greater than 10%, 33%, 50%, 90%, 95% or 99% as compared to a cell or organism not treated according to the present invention. Lower doses of injected material and longer times after administration of probe may result in inhibition in a smaller fraction of cells (e.g., at least 10%, 20%, 50%, 75%, 90%, or 95% of targeted cells). Quantitation of gene expression in a cell may show similar amounts of inhibition at the level of accumulation of target mRNA or translation of target protein. As an example, the efficiency of inhibition may be determined by assessing the amount of gene product in the cell: mRNA may be detected with a hybridization probe having a nucleotide sequence outside the region used for the inhibitory probe, or translated polypeptide may be detected with an antibody raised against the polypeptide sequence of that region. The cell with the target gene may be derived from or contained in any organism. The organism may a plant, animal, protozoan, bacterium, virus, or fungus. The plant may be a monocot, dicot or gymnosperm; the animal may be a vertebrate or invertebrate. Preferred microbes are those used in agriculture or by industry, and those that are pathogenic for plants or animals. Fungi include organisms in both the mold and yeast morphologies.

Plants include, but are not limited to: arabidopsis; field crops (e.g., alfalfa, barley, bean, corn, cotton, flax, pea, rape, rice, rye, safflower, sorghum, soybean, sunflower, tobacco, and wheat); vegetable crops (e.g., asparagus, beet, broccoli, cabbage, carrot, cauliflower, celery, cucumber, eggplant, lettuce, onion, pepper, potato, pumpkin, radish, spinach, squash, taro, tomato, and zucchini); fruit and nut crops (e.g., almond, apple, apricot, banana, blackberry, blueberry, cacao, cherry, coconut, cranberry, date, fajoa, filbert, grape, grapefruit, guava, kiwi, lemon, lime, mango, melon, nectarine, orange, papaya, passion fruit, peach, peanut, pear, pineapple, pistachio, plum, raspberry, strawberry, tangerine, walnut, and watermelon); and ornamentals (e.g., alder, ash, aspen, azalea, birch, boxwood, camellia, carnation, chrysanthemum, elm, fir, ivy, jasmine, juniper, oak, palm, poplar, pine, redwood, rhododendron, rose, and rubber).

Examples of vertebrate animals include fish, mammal, cattle, goat, pig, sheep, rodent, hamster, mouse, rat, primate, and human; invertebrate animals include nematodes, other worrns, drosophila, and other insects. Representative generae of nematodes include those that infect animals (e.g., Ancylostoma, Ascaridia, Ascaris, Bunostomum, Caenorhabditis, Capillaria, Chabertia, Cooperia, Dictyocaulus, Haemonchus, Heterakis, Nematodirus, Oesophagostomum, Ostertagia, Oxyaris, Parascaris, Strongylus, Toxascaris, Trichuris, Trichostrongylus, Tfhchonema, Toxocara, Uncinaria) and those that infect plants (e.g., Bursaphalenchus, Criconemella, Diiylenchus, Ditylenchus, Globodera, Helicotylenchus, Heterodera, Longidorus, Melodoigyne, Nacobbus, Paratylenchus, Pratylenchus, Radopholus, Rotelynchus, Tylenchus, and Xiphinema). Representative orders of insects include Coleoptera, Diptera, Lepidoptera, and Homoptera.

The cell having the target gene may be from the germ line or somatic, totipotent or pluripotent, dividing or non-dividing, parenchyma or epithelium, immortalized or transformed, or the like. The cell may be a stem cell or a differentiated cell. Cell types that are differentiated include adipocytes, fibroblasts, myocytes, cardiomyocytes, endothelium, neurons, glia, blood cells, megakaryocytes, lymphocytes, macrophages, neutrophils, eosinophils, basophils, mast cells, leukocytes, granulocytes, keratinocytes, chondrocytes, osteoblasts, osteoclasts, hepatocytes, and cells of the endocrine or exocrine glands.

The present invention may be used to introduce the probe into a cell for the treatment or prevention of disease. For example, the probe may be introduced into a cancerous cell or tumor and thereby inhibit gene expression of a gene required for maintenance of the carcinogenic/tumorigenic phenotype. To prevent a disease or other pathology, a target gene may be selected which is required for initiation or maintenance of the disease/pathology. Treatment would include amelioration of any symptom associated with the disease or clinical indication associated with the pathology.

A gene derived from any pathogen may be targeted for inhibition. For example, the gene could cause immunosuppression of the host directly or be essential for replication of the pathogen, transmission of the pathogen, or maintenance of the infection. The inhibitory probe could be introduced in cells in vitro or ex vivo and then subsequently placed into an animal to affect therapy, or directly treated by in vivo administration. A method of gene therapy can be envisioned. For example, cells at risk for infection by a pathogen or already infected cells, particularly human immunodeficiency virus (HIV) infections, may be targeted for treatment by introduction of probe according to the invention. The target gene might be a pathogen or host gene responsible for entry of a pathogen into its host, drug metabolism by the pathogen or host, replication or integration of the pathogen's genome, establishment or spread of an infection in the host, or assembly of the next generation of pathogen. Methods of prophylaxis (i.e., prevention or decreased risk of infection), as well as reduction in the frequency or severity of symptoms associated with infection, can be envisioned.

The present invention can be used for treatment or development of treatments for cancers of any type, including solid tumors and leukemias, including: apudoma, choristoma, branchioma, malignant carcinoid syndrome, carcinoid heart disease, carcinoma (e.g., Walker, basal cell, basosquamous, Brown-Pearce, ductal, Ehrlich tumor, in situ, Krebs 2, Merkel cell, mucinous, non-small cell lung, oat cell, papillary, scirrhous, bronchiolar, bronchogenic, squamous cell, and transitional cell), histiocytic disorders, leukemia (e.g., B cell, mixed cell, null cell, T cell, T-cell chronic, HTLV-II-associated, lymphocytic acute, lymphocytic chronic, mast cell, and myeloid), histiocytosis malignant, Hodgkin disease, immunoproliferative small, non-Hodgkin lymphoma, plasmacytoma, reticuloendotheliosis, melanoma, chondroblastoma, chondroma, chondrosarcoma, fibroma, fibrosarcoma, giant cell tumors, histiocytoma, lipoma, liposarcoma, mesothelioma, myxoma, myxosarcoma, osteoma, osteosarcoma, Ewing sarcoma, synovioma, adenofibroma, adenolymphoma, carcinosarcoma, chordoma, cranio-pharyngioma, dysgerminoma, hamartoma, mesenchymoma, mesonephroma, myosarcoma, ameloblastoma, cementoma, odontoma, teratoma, thymoma, trophoblastic tumor, adenocarcinoma, adenoma, cholangioma, cholesteatoma, cylindroma, cystadenocarcinoma, cystadenoma, granulosa cell tumor, gynandroblastoma, hepatoma, hidradenoma, islet cell tumor, Leydig cell tumor, papilloma, Sertoli cell tumor, theca cell tumor, leiomyoma, leiomyosarcoma, myoblastoma, myoma, myosarcoma, rhabdomyoma, rhabdomyosarcoma, ependymoma, ganglioneuroma, glioma, medulioblastoma, meningioma, neurilemmoma, neuroblastoma, neuroepithelioma, neurofibroma, neuroma, paraganglioma, paraganglioma nonchromaffin, angiokeratoma, angiolymphoid hyperplasia with eosinophilia, angioma sclerosing, angiomatosis, glomangioma, hemangioendothelioma, hemangioma, hemangiopericytoma, hemangiosarcoma, lymphangioma, lymphangiomyoma, lymphangiosarcoma, pinealoma, carcinosarcoma, chondrosarcoma, cystosarcoma phyllodes, fibrosarcoma, hemangiosarcoma, leiomyosarcoma, leukosarcoma, liposarcoma, lymphangiosarcoma, myosarcoma, myxosarcoma, ovarian carcinoma, rhabdomyosarcoma, sarcoma (e.g., Ewing, experimental, Kaposi, and mast cell), neoplasms (e.g., bone, breast, digestive system, colorectal, liver, pancreatic, pituitary, testicular, orbital, head and neck, central nervous system, acoustic, pelvic, respiratory tract, and urogenital), neurofibromatosis, and cervical dysplasia, and for treatment of other conditions in which cells have become immortalized or transformed. The invention could be used in combination with other treatment modalities, such as chemotherapy, cryotherapy, hyperthermia, radiation therapy, and the like.

As disclosed herein, the present invention is not limited to any type of target gene or nucleotide sequence. But the following classes of possible target genes are listed for illustrative purposes: developmental genes (e.g., adhesion molecules, cyclin dependent kinase inhibitors, Wnt family members, Pax family members, Winged helix family members, Hox family members, cytokines/lymphokines and their receptors, growth/differentiation factors and their receptors, neurotransmitters and their receptors); oncogenes (e.g., ABL1, BCL1, BCL2, BCL6, CBFA2, CBL, CSF1R, ERBA, ERBB, EBRB2, ETS1, ETS1, ETV6, FGR, FOS, FYN, HCR, HRAS, JUN, KRAS, LCK, LYN, MDM2, MLL, MYB, MYC, MYCL1, MYCN, NRAS, PIMI, PML, RET, SRC, TAL1, TCL3, and YES); tumor suppressor genes (e.g., APC, BRCA1, BRCA2, MADH4, MCC, NF1, NF2, RB1, TP53, and WT1); and enzymes (e.g., ACC synthases and oxidases, ACP desaturases and hydroxylases, ADP-glucose pyrophorylases, ATPases, alcohol dehydrogenases, amylases, amyloglucosidases, catalases, cellulases, chalcone synthases, chitinases, cyclooxygenases, decarboxylases, dextrinases, DNA and RNA polymerases, galactosidases, glucanases, glucose oxidases, granule-bound starch synthases, GTPases, helicases, hemicellulases, integrases, inuhinases, invertases, isomerases, kinases, lactases, lipases, lipoxygenases, lysozymes, nopaline synthases, octopine synthases, pectinesterases, peroxidases, phosphatases, phospholipases, phosphorylases, phytases, plant growth regulator synthases, polygalacturonases, proteinases and peptidases, pullanases, recombinases, reverse transcriptases, RUBISCOs, topoisomerases, and xylanases).

The present invention can comprise a method for producing plants with reduced susceptibility to climatic injury, susceptibility to insect damage, susceptibility to infection by a pathogen, or altered fruit ripening characteristics. The targeted gene may be an enzyme, a plant structural protein, a gene involved in pathogenesis, or an enzyme that is involved in the production of a non-proteinaceous part of the plant (i.e., a carbohydrate or lipid). If an expression construct is used to transcribe the RNA in a plant, transcription by a wound- or stress-inducible; tissue-specific (e.g., fruit, seed, anther, flower, leaf, root); or otherwise regulatable (e.g., infection, light, temperature, chemical) promoter may be used. By inhibiting enzymes at one or more points in a metabolic pathway or genes involved in pathogenesis, the effect may be enhanced: each activity will be affected and the effects may be magnified by targeting multiple different components. Metabolism may also be manipulated by inhibiting feedback control in the pathway or production of unwanted metabolic byproducts.

The present invention may be used to reduce crop destruction by other plant pathogens such as arachnids, insects, nematodes, protozoans, bacteria, or fungi. Some such plants and their pathogens are listed in Index of plant Diseases in the United States (U.S. Dept. of Agriculture Handbook No. 165, 1960); Distribution of Plant-Parasitic Nematode Species in North America (Society of Nematologists, 1985); and Fungi on Plants and Plant Products in the United States (American Phytopathological Society, 1989). Insects with reduced ability to damage crops or improved ability to prevent other destructive insects from damaging crops may be produced. Furthermore, some nematodes are vectors of plant pathogens, and may be attacked by other beneficial nematodes, which have no effect on plants. Inhibition of target gene activity could be used to delay or prevent entry into a particular developmental step (e.g., metamorphosis), if plant disease was associated with a particular stage of the pathogen's life cycle. Interactions between pathogens may also be modified by the invention to limit crop damage. For example, the ability of beneficial nematodes to attack their harmful prey may be enhanced by inhibition of behavior-controlling nematode genes according to the invention.

Although pathogens cause disease, some of the microbes interact with their plant host in a beneficial manner. For example, some bacteria are involved in symbiotic relationships that fix nitrogen and some fungi produce phytohormones. Such beneficial interactions may be promoted by using the present invention to inhibit target gene activity in the plant and/or the microbe.

Another utility of the present invention is a method of identifying gene function in an organism comprising the use of probes to inhibit the activity of a target gene of previously unknown function. Instead of the time consuming and laborious isolation of mutants by traditional genetic screening, functional genomics would envision determining the function of uncharacterized genes by employing the invention to reduce the amount and/or alter the timing of target gene activity. The invention could be used in determining potential targets for pharmaceutics, understanding normal and pathological events associated with development, determining signaling pathways responsible for postnatal development/aging, and the like. The increasing speed of acquiring nucleotide sequence information from genomic and expressed gene sources, including total sequences for the yeast, D. melanogaster, and C. elegans genomes, can be coupled with the invention to determine gene function in an organism (e.g., nematode). The preference of different organisms to use particular codons, searching sequence databases for related gene products, correlating the linkage map of genetic traits with the physical map from which the nucleotide sequences are derived, and artificial intelligence methods may be used to define putative open reading frames from the nucleotide sequences acquired in such sequencing projects.

A simple assay would be to inhibit gene expression according to the partial sequence available from an expressed sequence tag (EST). Functional alterations in growth, development, metabolism, disease resistance, or other biological processes would be indicative of the normal role of the EST's gene product.

Probes of the invention can be introduced into an intact cell/organism containing the target gene, allowing the present invention to be used in high throughput screening (HTS). For example, probes can be produced by an amplification reaction using primers flanking the inserts of any gene library derived from the target cell/organism. Inserts may be derived from genomic DNA or mRNA (e.g., cDNA and cRNA). Individual clones from the library can be replicated and then isolated in separate reactions, but preferably the library is maintained in individual reaction vessels (e.g., a 96-well microtiter plate) to minimize the number of steps required to practice the invention and to allow automation of the process. Solutions containing probes that are capable of inhibiting the different expressed genes can be placed into individual wells positioned on a microtiter plate as an ordered array, and intact cells/organisms in each well can be assayed for any changes or modifications in behavior or development due to inhibition of target gene activity. The amplified probes can be fed directly to, injected into, the cell/organism containing the target gene. Alternatively, the probes can be produced by in vivo or in vitro transcription from an expression construct used to produce the library. The construct can be replicated as individual clones of the library and transcribed to produce the probes; each clone can then be fed to, or injected into, the cell/organism containing the target gene. The function of the target gene can be assayed from the effects it has on the cell/organism when gene activity is inhibited. This screening could be amenable to small subjects that can be processed in large number, for example: arabidopsis, bacteria, drosophila, fungi, nematodes, viruses, zebrafish, and tissue culture cells derived from mammals.

A nematode or other organism that produces a calorimetric, fluorogenic, or luminescent signal in response to a regulated promoter (e.g., transfected with a reporter gene construct) can be assayed in an HTS format to identify DNA-binding proteins that regulate the promoter. In the assay's simplest form, inhibition of a negative regulator results in an increase of the signal and inhibition of a positive regulator results in a decrease of the signal.

If a characteristic of an organism is determined to be genetically linked to a polymorphism through RFLP or QTL analysis, the present invention can be used to gain insight regarding whether that genetic polymorphism might be directly responsible for the characteristic. For example, a fragment defining the genetic polymorphism or sequences in the vicinity of such a genetic polymorphism can be amplified to produce an RNA, the probe can be introduced to the organism, and whether an alteration in the characteristic is correlated with inhibition can be determined. Of course, there may be trivial explanations for negative results with this type of assay, for example: inhibition of the target gene causes lethality, inhibition of the target gene may not result in any observable alteration, the fragment contains nucleotide sequences that are not capable of inhibiting the target gene, or the target gene's activity is redundant.

The present invention may be useful in allowing the inhibition of essential genes. Such genes may be required for cell or organism viability at only particular stages of development or cellular compartments. The functional equivalent of conditional mutations may be produced by inhibiting activity of the target gene when or where it is not required for viability. The invention allows addition of the probe at specific times of development and locations in the organism without introducing permanent mutations into the target genome.

If alternative splicing produced a family of transcripts that were distinguished by usage of characteristic exons, the present invention can target inhibition through the appropriate exons to specifically inhibit or to distinguish among the functions of family members. For example, a hormone that contained an alternatively spliced transmembrane domain may be expressed in both membrane bound and secreted forms. Instead of isolating a nonsense mutation that terminates translation before the transmembrane domain, the functional consequences of having only secreted hormone can be determined according to the invention by targeting the exon containing the transmembrane domain and thereby inhibiting expression of membrane-bound hormone.

The present invention may be used alone or as a component of a kit having at least one of the reagents necessary to carry out the in vitro or in vivo introduction of the probe to test samples or subjects. Preferred components are the probe and a vehicle that promotes introduction of the probe. Such a kit may also include instructions to allow a user of the kit to practice the invention.

Pesticides may include the probe itself, an expression construct capable of expressing the probe, or organisms transfected with the expression construct. The pesticide of the present invention may serve as an arachnicide, insecticide, nematicide, viricide, bactericide, and/or fungicide. For example, plant parts that are accessible above ground (e.g., flowers, fruits, buds, leaves, seeds, shoots, bark, stems) may be sprayed with pesticide, the soil may be soaked with pesticide to access plant parts growing beneath ground level, or the pest may be contacted with pesticide directly. If pests interact with each other, the probe may be transmitted between them. Alternatively, if inhibition of the target gene results in a beneficial effect on plant growth or development, the aforementioned probe, expression construct, or transfected organism may be considered a nutritional agent. In either case, genetic engineering of the plant is not required to achieve the objectives of the invention.

Alternatively, an organism may be engineered to produce the probe, which produces commercially or medically beneficial results, for example, resistance to a pathogen or its pathogenic effects, improved growth, or novel developmental patterns.

Used as either a pesticide or nutrient, a formulation of the present invention may be delivered to the end user in dry or liquid form: for example, as a dust, granulate, emulsion, paste, solution, concentrate, suspension, or encapsulation. Instructions for safe and effective use may also be provided with the formulation. The formulation might be used directly, but concentrates would require dilution by mixing with an extender provided by the formulator or the end user. Similarly, an emulsion, paste, or suspension may require the end user to perform certain preparation steps before application. The formulation may include a combination of chemical additives known in the art such as solid carriers, minerals, solvents, dispersants, surfactants, emulsifiers, tackifiers, binders, and other adjuvants. Preservatives and stabilizers may also be added to the formulation to facilitate storage. The crop area or plant may also be treated simultaneously or separately with other pesticides or fertilizers. Methods of application include dusting, scattering or pouring, soaking, spraying, atomizing, and coating. The precise physical form and chemical composition of the formulation, and its method of application, would be chosen to promote the objectives of the invention and in accordance with prevailing circumstances. Expression constructs and transfected hosts capable of replication may also promote the persistence and/or spread of the formulation.

The invention can be used to induce cell apoptosis or necrosis.

Probes of the invention are preferably 2 to 200 bases long (more preferably 5 to 30 bases long), and can be single or double-stranded. Thus, suitable probes for use in the inventive assay include, e.g., ssDNA, RNA, ssPNA, LNA, dsDNA, dsRNA, DNA:RNA hybrids, dsPNA, PNA:DNA hybrids and other single and double-stranded nucleic acids and nucleic acid analogues having uncharged, partially-charged, sugar phosphate and/or peptide backbones. The length of the probe can be selected to match the length of the target.

Probes of the invention are preferably safe to use and stable for years. Accordingly, probes can be made or ordered in large quantities and stored.

The reliability of the invention is independent of guanine and cytosine content in either the probe or the target. In the traditional W-C motif, since G:C base pairs form three hydrogen bonds, while A:T base pairs form only two hydrogen bonds, target and probe sequences with a higher G or C content are more stable, possessing higher melting temperatures. Consequently, base pair mismatches that increase the GC content of the hybridized probe and target region above that present in perfectly matched hybrids may offset the binding weakness associated with a mismatched probe.

The ratio of probe to target is preferably about 1:1 to about 1000:1.

We have noticed that duplexes which complex parallel strands of nucleic acid containing complementary base sequences bind to form triplexes at a different rate and bind as a culmination of a very different process than do bases in a double helix formed by nucleic acid strands of opposite directionality. Strands of opposite directionality (i.e., antiparallel strands) readily present regularly spaced bases in a planar orientation to the bases opposite with minimal backbone distortion.

The various complexes of the invention comprise a probe containing a heteropolymeric probe sequence of nucleobases and/or nucleobase analogues, and a target containing a heteropolymeric target sequence of nucleobases and/or nucleobase analogues. The complex is synthetic or purified in that at least one of either the probe or the target is synthetic or purified. The backbone of the probe is a deoxyribose phosphate backbone such as in DNA, or a peptide-like backbone such as in PNA, or is of some other uncharged or partially charged (negatively or positively) moieties.

In certain embodiments, the probe and target are single-stranded and the complex is a duplex. When said probe and target are a duplex they have parallel directionality with W-C complementary or homologous binding, or have antiparallel directionality with homologous binding.

In other embodiments, either the probe or the target is single-stranded and the other of said probe or target is double-stranded and the resulting complex is a triplex. This complex can be free of PNA.

In certain embodiments, the triplex contains a heteropolymeric probe sequence parallel to a heteropolymeric target sequence, wherein the heteropolymeric probe sequence is bonded to the heteropolymeric target sequence by homologous base binding or Watson-Crick complementary base binding. In certain other embodiments, the heteropolymeric probe sequence is antiparallel to the heteropolymeric target sequence and the heteropolymeric probe sequence is bonded to the heteropolymeric target sequence by homologous base binding or Watson-Crick complementary base binding.

In certain embodiments of the triplex complex, the target includes a first strand containing a heteropolymeric target sequence and a second strand containing a second heteropolymeric target sequence that is Watson-Crick complementary and antiparallel to the first heteropolymeric target sequence. The heteropolymeric probe sequence is bonded to the first heteropolymeric target sequence by homologous base bonding and is also bonded to the second heteropolymeric target sequence by Watson-Crick complementary base bonding.

In certain other embodiments of the triplex complex, the target includes a first strand containing a heteropolymeric target sequence and a second strand containing a second heteropolymeric target sequence that is Watson-Crick complementary and antiparallel to the first heteropolymeric target sequence. The heteropolymeric probe sequence is bonded to the first heteropolymeric target sequence by Watson-Crick complementary base bonding and is also bonded to the second heteropolymeric target sequence by homologous base bonding.

In certain embodiments, the probe and the target are double-stranded and the resulting complex is a quadruplex. This complex can be free of PNA.

In certain embodiments, the quadruplex contains a heteropolymeric probe sequence parallel or antiparallel to a heteropolymeric target sequence, wherein the heteropolymeric probe sequence is bonded to the heteropolymeric target sequence by homologous base binding or Watson-Crick complementary base binding. In such embodiments, the quadruplex complex contains a first probe strand containing said heteropolymeric probe sequence and a second probe strand containing a second heteropolymeric probe sequence that is complementary and antiparallel to the first probe sequence. The target includes a first target strand containing a heteropolymeric target sequence and a second target strand containing a second heteropolymeric target sequence that is complementary and antiparallel to the first.

In such quadruplex embodiments, the heteropolymeric probe sequence can bond to the heteropolymeric target sequence by Watson-Crick complementary or homologous base binding and the heteropolymeric probe sequence can optionally and additionally bond to the second heteropolymeric target sequence by homologous or Watson-Crick complementary base binding, respectively. Thus, when the heteropolymeric probe sequence bonds to the heteropolymeric target sequence by homologous base bonding, the heteropolymeric probe sequence optionally bonds to the second heteropolymeric target sequence by Watson-Crick complementary base bonding, and when the heteropolymeric probe sequence bonds to the heteropolymeric target sequence by Watson-Crick complementary base bonding, the heteropolymeric probe sequence optionally bonds to the second heteropolymeric target sequence by homologous base bonding.

In certain embodiments, homologous quadruplexes act as a signal for methylation of cytosines, leading to the formation of heterochromatin in the vicinity. See Aufsatz et al., “RNA-directed DNA methylation in Arabidopsis,” Proc Natl Acad Sci USA Dec. 10, 2002;99 Suppl 4:16499-506.

The invention will be illustrated in more detail with reference to the following Examples demonstrating the specific binding capabilities of nucleic acids upon which the invention depends, but it should be understood that the present invention is not deemed to be limited thereto.

### EXAMPLES

### Example 1

Complementary sense and antisense 50-mer ssDNA target sequences, derived from exon 10 of the human cystic fibrosis gene (Nature 380, 207 (1996)) were synthesized on a DNA synthesizer (Expedite 8909, PerSeptive Biosystems) and purified by HPLC. SsDNA oligonucleotides were dissolved in ddH₂O and diluted to a concentration of 1 pmole/μl. Equimolar amounts of complementary oligonucleotides were heated at 95° C. for 10 min and allowed to anneal gradually in the presence of 10 mM Tris, pH 7.5, 1 mM EDTA and 100 mM NaCl, as the temperature cooled to 21° C. over 1.5 hours. DsDNA oligonucleotides were diluted in ddH₂O at a concentration of 1 pmole/μl.

The sequence for the sense strand of the wild-type target DNA (SEQ ID NO:1) was: 5′-TGG CAC CAT TAA AGA AAA TAT CAT CTT TGG TGT TTC CTA TGA TGA ATA TA-3′.

The sequence for the antisense strand of the wild-type target DNA (SEQ ID NO:1) was: 5′-TAT ATT CAT CAT AGG AAA CAC CAA AGA TGA TAT TTT CTT TAA TGG TGC CA-3′.

SEQ ID NO:2 was a 50-mer mutant dsDNA target sequence identical to wild-type target DNA (SEQ ID NO:1) except for a one base pair mutation (underlined) at amino acid position 507 at which the wild-type sense strand sequence CAT was changed to CGT.

The sequence for the sense strand of SEQ ID NO:2 was: 5′-TGG CAC CAT TAA AGA AAA TAT CGT CTT TGG TGT TTC CTA TGA TGA ATA TA-3′.

The sequence for the antisense strand of SEQ ID NO:2 was: 5′-TAT ATT CAT CAT AGG AAA CAC CAA AGA CGA TAT TTT CTT TAA TGG TGC CA-3′.

SEQ ID NO:3 was a 50-mer mutant dsDNA target sequence identical to wild-type target DNA (SEQ ID NO:1) except for a consecutive two base pair mutation (underlined) at amino acid positions 506 and 507 at which the wild-type sense strand sequence CAT was changed to ACT.

The sequence for the sense strand of SEQ ID NO:3 was: 5′-TGG CAC CAT TAA AGA AAA TAT ACT CTT TGG TGT TTC CTA TGA TGA ATA TA-3′.

The sequence for the antisense strand of SEQ ID NO:3 was: 5′-TAT ATT CAT CAT AGG AAA CAC CAA AGA GTA TAT TTT CTT TAA TGG TGC CA-3′.

SEQ ID NO:4 was a 50-mer mutant dsDNA target sequence identical to wild-type target DNA (SEQ ID NO:1) except for a consecutive three base pair mutation (underlined) at amino acid positions 506 and 507 at which the wild-type sense strand sequence CAT was changed to ACG.

The sequence for the sense strand of SEQ ID NO:4 was: 5′-TGG CAC CAT TAA AGA AAA TAT ACG CTT TGG TGT TTC CTA TGA TGA ATA TA-3′.

The sequence for the antisense strand of SEQ ID NO:4 was: 5′-TAT ATT CAT CAT AGG AAA CAC CAA AGCGTA TAT TTT CTT TAA TGG TGC CA-3′.

SEQ ID NO:5 was a 50-mer dsDNA target sequence modified from SEQ ID NO:1, wherein the percent GC content was changed from 30% to 52%.

The sequence for the sense strand of the wild-type target DNA (SEQ ID NO:5) was: 5′-GAG CAC CAT GAC AGA CAC TGT CAT CTC TGG TGT GTC CTA CGA TGA CTC TG-3′.

The sequence for the antisense strand of the wild-type target DNA (SEQ ID NO:5) was: 5′-CAG AGT CAT CGT AGG ACA CAC CAG AGA TGA CAG TGT CTG TCA TGG TGC TC-3′.

SEQ ID NO:6 was a 50-mer mutant dsDNA target sequence identical to SEQ ID NO:5, except for a one base pair mutation (underlined), at which the sense strand sequence CAT was changed to CGT.

The sequence for the sense strand of mutant SEQ ID NO:6 was: 5′-GAG CAC CAT GAC AGA CAC TGT CGT CTC TGG TGT GTC CTA CGA TGA CTC TG-3′.

The sequence for the antisense strand of mutant SEQ ID NO:6 was: 5′-CAG AGT CAT CGT AGG ACA CAC CAG AGA CGA CAG TGT CTG TCA TGG TGC TC-3′.

SEQ ID NO:7 was a 50-mer mutant dsDNA target sequence identical to SEQ ID NO:5, except for a one base pair mutation (underlined), at which the sense strand sequence CTC was changed to CTT.

The sequence for the sense strand of mutant SEQ ID NO:7 was: 5′-GAG CAC CAT GAC AGA CAC TGT CAT CTT TGG TGT GTC CTA CGA TGA CTC TG-3′.

The sequence for the antisense strand of mutant SEQ ID NO:7 was: 5′-CAG AGT CAT CGT AGG ACA CAC CAA AGA TGA CAG TGT CTG TCA TGG TGC TC-3′.

SEQ ID NO:8 was a 50-mer mutant dsDNA target sequence identical to SEQ ID NO:5, except for a consecutive two base pair mutation (underlined), at which the sense strand sequence CAT was changed to ACT.

The sequence for the sense strand of mutant SEQ ID NO:8 was: 5′-GAG CAC CAT GAC AGA CAC TGT ACT CTC TGG TGT GTC CTA CGA TGA CTC TG-3′.

The sequence for the antisense strand of mutant SEQ ID NO:8 was: 5′-CAG AGT CAT CGT AGG ACA CAC CAG AGA GTA CAG TGT CTG TCA TGG TGC TC-3′.

SEQ ID NO:9 was a 47-mer mutant dsDNA target sequence identical to wild-type target DNA (SEQ ID NO:1) except for a consecutive three base pair deletion (indicated by three dots) at amino acid positions 507 and 508 at which the wild-type sense strand sequence CTT is deleted.

The sequence for the sense strand of SEQ ID NO:9 was: 5′-TGG CAC CAT TAA AGA AAA TAT CAT . . . TGG TGT TTC CTA TGA TGA ATA TA-3′.

The sequence for the antisense strand of SEQ ID NO:9 was: 5′-TAT ATT CAT CAT AGG AAA CAC CA . . . A TGA TAT TTT CTT TAA TGG TGC CA-3′.

SEQ ID NO:10 was a 50-mer mutant dsDNA target sequence identical to SEQ ID NO:5, except for a one base pair mutation (underlined), at which the sense strand sequence CAT was changed to CTT.

The sequence for the sense strand of mutant SEQ ID NO:10 was: 5′-GAG CAC CAT GAC AGA CAC TGT CTT CTC TGG TGT GTC CTA CGA TGA CTC TG-3′.

The sequence for the antisense strand of mutant SEQ ID NO:10 was: 5′-CAG AGT CAT CGT AGG ACA CAC CAG AGA AGA CAG TGT CTG TCA TGG TGC TC-3′.

SEQ ID NO:11 was a 50-mer mutant dsDNA target sequence identical to SEQ ID NO:5, except for a one base pair mutation (underlined), at which the sense strand sequence CTC was changed to CCC.

The sequence for the sense strand of mutant SEQ ID NO:11 was: 5′-GAG CAC CAT GAC AGA CAC TGT CAT CCC TGG TGT GTC CTA CGA TGA CTC TG-3′.

The sequence for the antisense strand of mutant SEQ ID NO:11 was: 5′-CAG AGT CAT CGT AGG ACA CAC CAG GGA TGA CAG TGT CTG TCA TGG TGC TC-3′.

The PNA probes were synthesized, HPLC purified and confirmed by mass spectroscopy by Commonwealth Biotechnologies, Inc. (Richmond, Va., USA). PNA probes were first dissolved in 0.1% TFA (trifluoroacetic acid) to a concentration of 10 mg/ml, and then diluted to 1 mg/ml by the addition of ddH₂O. Final PNA stock solutions were prepared in ddH₂O at a concentration of 1 pmole/μl.

Probe No. 1 was a 15-mer PNA probe designed to be completely complementary to a 15 nucleotide segment of the sense strand of the 50-mer wild-type target DNA (SEQ ID NO:1), overlapping amino acid positions 505 to 510 (Nature 380, 207 (1996)). The directionality of the probe was opposite or antiparallel to that of the sense strand in the target.

The sequence for Probe No. 1 (SEQ ID NO:12) was: 5′-H-CAC CAA AGA TGA TAT-Lys-CONH₂-3′.

Probe No. 2 was a 15-mer PNA probe identical in sequence to Probe No. 1, but was of the same directionality, or parallel to that of the sense strand in the dsDNA target.

The sequence for Probe No. 2 (SEQ ID NO:13) was: 5′-H-TAT AGT AGA AAC CAC-Lys-CONH₂-3′.

The 15-mer ssDNA probes were synthesized and purified by HPLC as above. SsDNA probes were dissolved in ddH₂O at a concentration of 1 pmole/μl.

Probe No. 3 was a 15-mer ssDNA probe designed to be completely complementary to a 15 nucleotide segment of the sense strand of the 50-mer wild-type target DNA (SEQ ID NO:5). The directionality of the probe was opposite or antiparallel to that of the sense strand in the target.

The sequence for Probe No. 3 (SEQ ID NO:14) was: 5′-CAC CAG AGA TGA CAG-3′.

Probe No. 4 was a 15-mer ssDNA probe identical in sequence to Probe No. 3, but was of the same directionality, or parallel to that of the sense strand in the dsDNA target.

The sequence for Probe No. 4 (SEQ ID NO:15) was: 5′-GAC AGT AGA GAC CAC-3′.

Probe No. 5 was a 15-mer antiparallel ssDNA probe identical to Probe No. 3, except it had an attached fluorescein moiety at the 5′ position.

The sequence for Probe No. 5 (SEQ ID NO:16) was: 5′-Flu-CAC CAG AGA TGA CAG-3′.

Probe No. 6 was a 15-mer parallel ssDNA probe identical to Probe No. 4, except it had an attached fluorescein moiety at the 5′ position.

The sequence for Probe No. 6 (SEQ ID NO:17) was: 5′-Flu-GAC AGT AGA GAC CAC-3′.

Probe No. 7 was a 15-mer ssDNA probe, with an attached fluorescein moiety at the 5′ position, designed to be completely complementary to a 15 nucleotide segment of the sense strand of the 50-mer wild-type target DNA (SEQ ID NO:1). The directionality of the probe was opposite or antiparallel to that of the sense strand in the target.

The sequence for Probe No. 7 (SEQ ID NO:18) was: 5′-Flu-CAC CAA AGA TGA TAT-3′.

Probe No. 8 was a 15-mer ssDNA probe designed to be completely complementary to a 15 nucleotide segment of the sense strand of the 50-mer wild-type target DNA (SEQ ID NO:1). The directionality of the probe was antiparallel to that of the sense strand in the target.

The sequence for Probe No. 8 (SEQ ID NO:19) was: 5′-CAC CAA AGA TGA TAT-3′.

Probe No. 9 and Probe No. 10 were 15-mer mutant ssDNA probes identical in sequence to wild-type Probe No. 8, except for a one base mutation (underlined).

The sequence for Probe No. 9 (SEQ ID NO:20) was: 5′-CAC GAA AGA TGA TAT-3′.

The sequence for Probe No. 10 (SEQ ID NO:21) was: 5′-CAC CAA ACA TGA TAT-3′.

It is well known that ssDNA strands of mixed base sequence readily form ssPNA:ssDNA duplexes on a Watson-Crick pairing basis when reacted with either antiparallel or parallel synthesized ssPNA strands at room temperature. We have previously shown that such ssPNA:ssDNA complexes containing perfectly matched sequences can reliably be distinguished from ssPNA:ssDNA complexes containing a 1 bp mismatch when assayed in the presence of the DNA intercalator, YOYO-1 (Molecular Probes, Eugene, Oreg., USA), and that the order of assembly of the PNA strand has a significant bearing on its ability to specifically bind a ssDNA target. Example 1 compares the efficiency of formation of dsDNA duplexes when wild-type or mutant ssDNA target sequences are reacted with Watson-Crick complementary antiparallel ssDNA probes or with homologous, that is to say identical parallel, ssDNA probes.

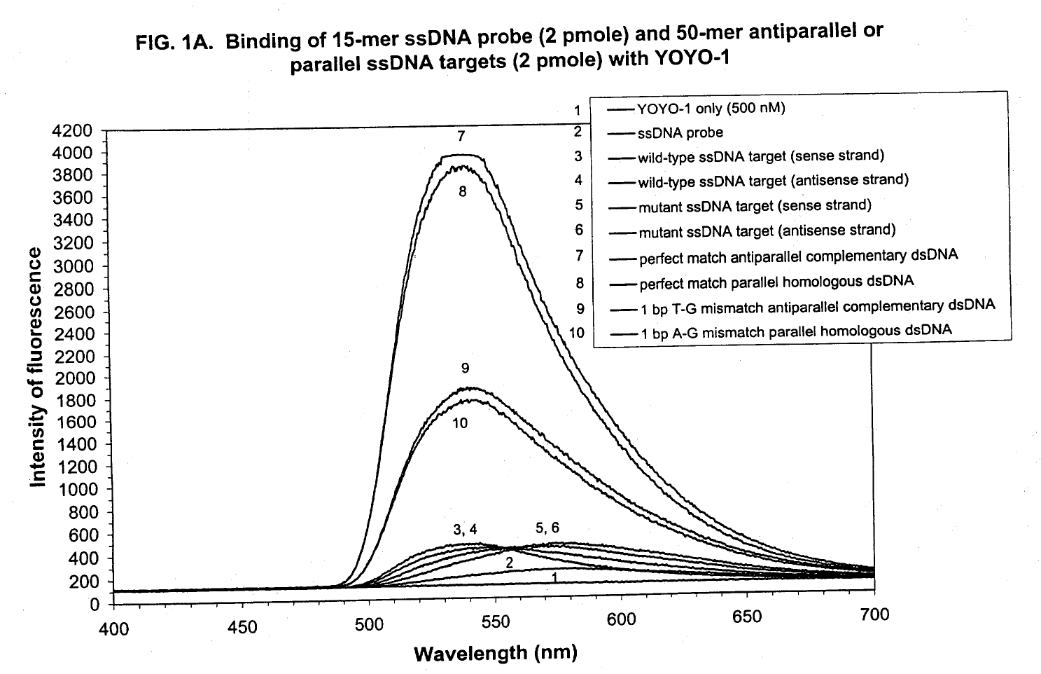

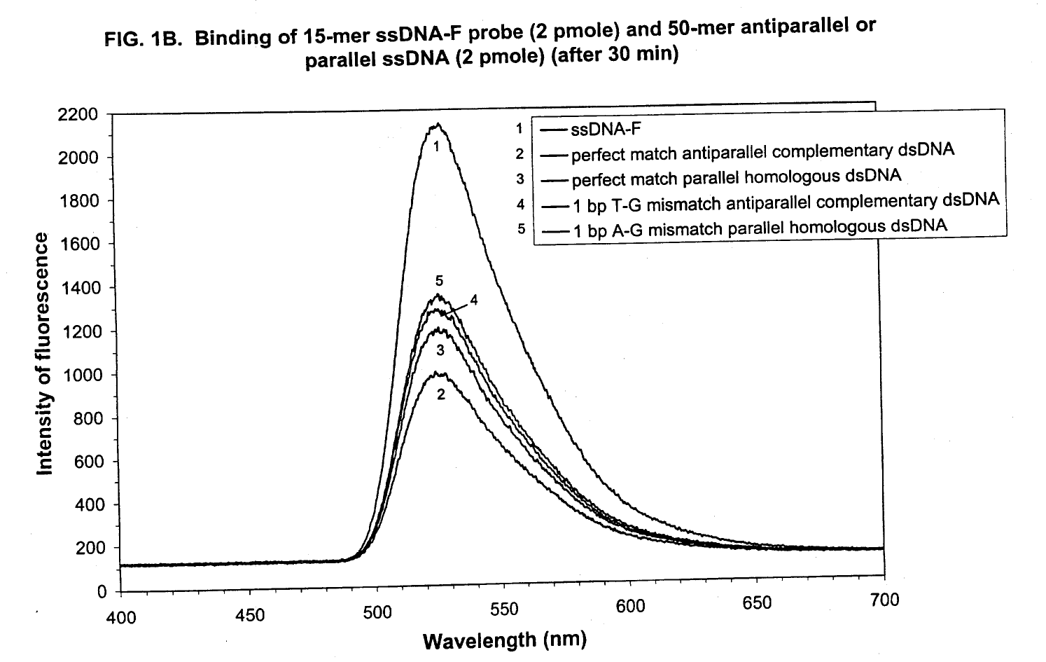

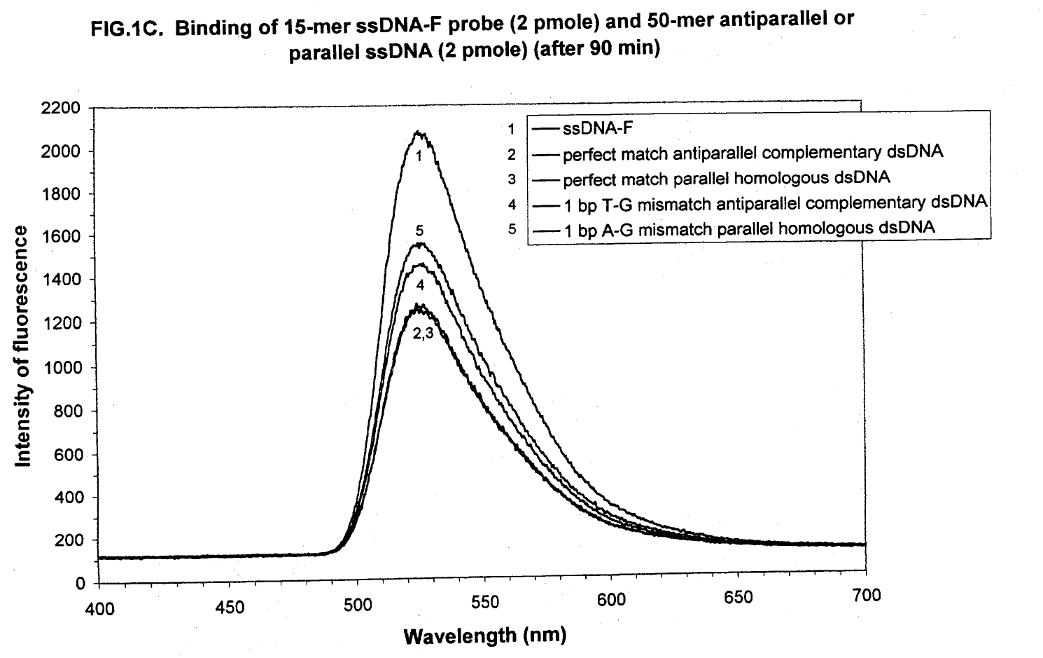

The hybridization reaction mixtures giving rise to the data illustrated in FIG. 1A, each contained the following mixture: 2 pmoles of ssDNA target, 2 pmoles of ssDNA probe, 0.5×TBE and 500 nM of YOYO-1 in a final volume of 40 μl. The reaction mixtures were incubated at room temperature (21° C.) for 5 minutes, placed into a quartz cuvette, irradiated with an argon ion laser beam having a wavelength of 488 nm and monitored for fluorescent emission. The intensity of fluorescence was plotted as a function of wavelength for each sample analyzed.

In FIGS. 1B and 1C, the hybridization reaction mixtures (40 μl) each contained the following: 2 pmoles of ssDNA target, 2 pmoles of 5′-fluorescein labeled ssDNA probe, 10 mM Tris-HCl, pH 7.5, and 1 mM EDTA. The reaction mixtures were incubated at room temperature (21° C.) for 30 minutes or 90 minutes. Following incubation, each sample was placed into a quartz cuvette, irradiated with an argon ion laser beam having a wavelength of 488 nm and monitored for fluorescent emission. The maximum fluorescent intensities occurred at a wavelength of 525 nm, the emission wavelength for fluorescein. The intensity of fluorescent emission was plotted as a function of wavelength for each sample analyzed.

When the ssDNA Probe No. 3 was reacted with the 50-mer wild-type sense strand of SEQ ID NO:5 or with the 50-mer mutant sense strand of SEQ ID NO:7 in the presence of YOYO-L, antiparallel complementary ssDNA:ssDNA duplexes were formed (FIG. 1A). The fluorescent intensity emitted by the 1 bp T-G mismatched antiparallel complementary duplex (sense strand of SEQ ID NO:7+Probe No. 3) was 56% lower than that obtained by the perfectly matched antiparallel complementary duplex (sense strand of SEQ ID NO:5+Probe No. 3).

When the ssDNA Probe No. 3 was reacted with the 50-mer wild-type antisense strand of SEQ ID NO:5 in the presence of YOYO-1, the efficiency of parallel homologous ssDNA:ssDNA duplex formation was only 3% lower than the efficiency of antiparallel complementary ssDNA:ssDNA duplex formation (FIG. 1A). This result was completely unanticipated. The 1 bp A-G mismatched parallel homologous duplex formed when the 50-mer mutant antisense strand of SEQ ID NO:7 was reacted with the ssDNA Probe No. 3 in the presence of YOYO-1, produced a fluorescent emission intensity that was 56% lower than that emitted by the perfectly parallel homologous duplex (FIG. 1A). Control samples comprising each 50-mer ssDNA target plus 500 nM YOYO-1 exhibited levels of fluorescence which ranged from 91% to 92% lower than that observed with the perfectly matched duplexes (FIG. 1A). The level of fluorescence emitted by the 15-mer ssDNA Probe No. 3 plus 500 nM YOYO-1 was slightly greater than that produced by YOYO-1 alone. The shift in fluorescent emission wavelength observed with the ssDNA targets and probe is typical of YOYO-1's emission profile in the presence of ssDNA.

YOYO-1 facilitated DNA complex formation between a ssDNA probe and a complementary base sequence in an antiparallel ssDNA target, or between a ssDNA probe and an identical base sequence in a parallel ssDNA target, with similar efficacy, to allow differentiation between perfectly matched complexes and those containing a 1 bp mismatch. In the parallel homologous complexes, the 1 bp mismatch was a non-homologous base pair.

The comparative efficiency of antiparallel complementary and parallel homologous dsDNA duplex formation was further examined using ssDNA targets and ssDNA-F probes in the absence of complex promoting agents such as YOYO-1 or cations. When the ssDNA-F Probe No. 5 was incubated for 30 minutes in Tris buffer at room temperature with the 50-mer wild-type sense strand of SEQ ID NO:5, the Watson-Crick complementary antiparallel ssDNA:ssDNA-F duplexes were formed very efficiently, resulting in a 53% reduction in fluorescent emission compared to that emitted by Probe No. 5 alone (FIG. 1B). By contrast, antiparallel complementary ssDNA:ssDNA-F complexes that contained a 1 bp T-G mismatch (sense strand of SEQ ID NO:7+Probe No. 5) were less stable, resulting in only a 40% decrease in fluorescent emission compared to that emitted by Probe No. 5 alone after a 30 minute incubation (FIG. 1B).

Parallel homologous ssDNA:ssDNA-F complexes were formed when the ssDNA Probe No. 5 was reacted with the 50-mer wild-type antisense strand of SEQ ID NO:5 or with the 50-mer mutant antisense strand of SEQ ID NO:7, generating fluorescent emission intensities that were 44% and 37% lower, respectively, than that emitted by ssDNA Probe No. 5 alone after a 30 minute incubation (FIG. 1B). The avid formation of parallel homologous ssDNA:ssDNA-F complexes in the absence of a promoting agent was completely unanticipated. The discrimination between signals emitted from perfectly matched duplexes and 1 bp mismatched duplexes in the absence of complex promoting agents, was not as dramatic as that observed when YOYO-1 was present and served as the promoter and signaling agent (compare FIGS. 1A and 1B). This was the case for both antiparallel and parallel duplexes. Slightly less discrimination between perfectly matched and 1 bp mismatched DNA complexes was observed when a parallel homologous ssDNA target was used than when an antiparallel complementary ssDNA target was used to produce the ssDNA:ssDNA-F complexes (FIG. 1B).

After a 90 minute incubation, Watson-Crick antiparallel dsDNA:ssDNA-F complexes consisting of perfectly complementary sequences (sense strand of SEQ ID NO:5+Probe No. 5) or 1 bp T-G mismatched sequences (sense strand of SEQ ID NO:7+Probe No. 5) produced a 39% and 30% decrease, respectively, in fluorescent emission intensity compared to that emitted by Probe No. 5 alone (FIG. 1C). Remarkably, parallel homologous ssDNA:ssDNA-F complexes exhibited the same level of stability after 90 minutes of incubation as did the Watson-Crick antiparallel ssDNA:ssDNA-F complexes. The fluorescent intensities for a perfectly parallel homologous duplex (antisense strand of SEQ ID NO:5+Probe No. 5) and a 1 bp A-G mismatched parallel homologous duplex (antisense strand of SEQ ID NO:7+Probe No. 5) were 40% and 25% lower, respectively, than that emitted by ssDNA Probe No. 5 alone after a 90 minute incubation (FIG. 1C).

The mechanism of recognition and binding of the homologous bases in the parallel dsDNA duplexes is unknown at this time. Nevertheless, recognition and binding of parallel homologous ssDNA sequences occurred in a configuration which allowed the discrimination between perfectly matched ssDNA:ssDNA complexes and those containing a 1 bp or 2 bp mismatch. In these parallel homologous complexes, the 1 bp mismatch was a non-homologous base pair.

### Example 2

In Example 1, the remarkable efficiency of parallel homologous ssDNA:ssDNA duplex formation was demonstrated both in the presence of a complex promoting agent such as YOYO-l and in the absence of any complex promoting agent. The recognition and binding of the homologous bases in the parallel dsDNA duplexes was such as to allow easy discrimination between perfectly homologous base sequences and parallel homologous sequences that contained a 1 bp mismatch. These parallel homologous 1 bp mismatches were also clearly recognizable as mismatches based on Watson-Crick complementary recognition and binding rules. Example 2 examines the recognition and binding efficiency of parallel homologous dsDNA duplexes that contain A-T or G-C base pairings, to determine whether these Watson-Crick complementary pairings appear as mismatches in a parallel homologous binding reaction.

Each hybridization reaction mixture (40 μl) contained the following: 2 pmoles of ssDNA target, 2 pmoles of ssDNA probe, 0.5×TBE and 500 nM of YOYO-1. The reaction mixtures were incubated at room temperature (21° C.) for 5 minutes, placed into a quartz cuvette, irradiated with an argon ion laser beam having a wavelength of 488 nm and monitored for fluorescent emission. The intensity of fluorescence was plotted as a function of wavelength for each sample analyzed.

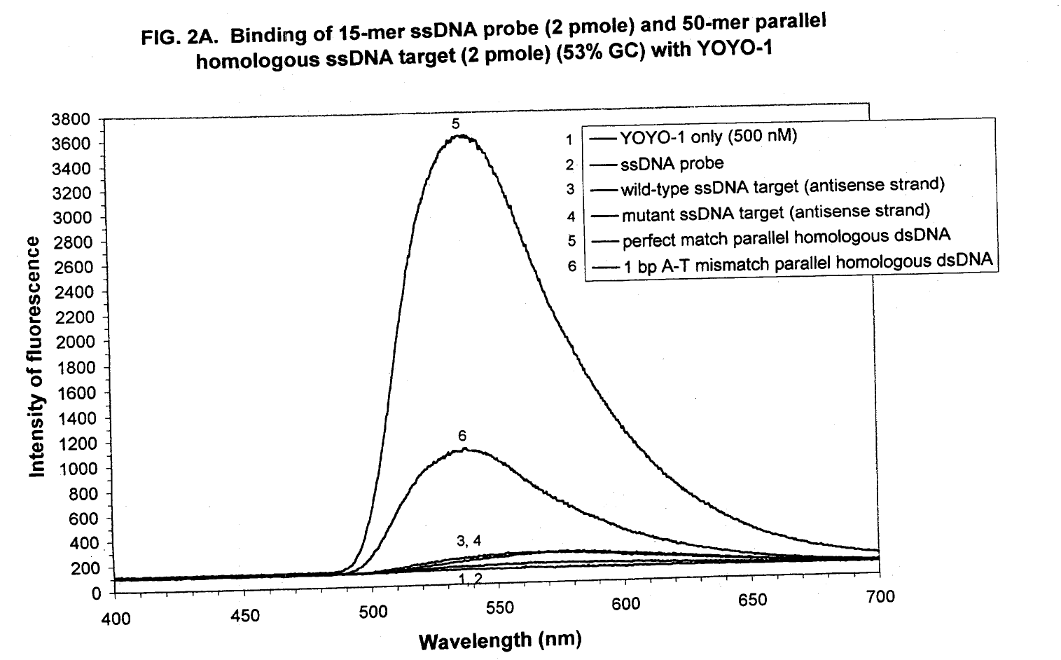

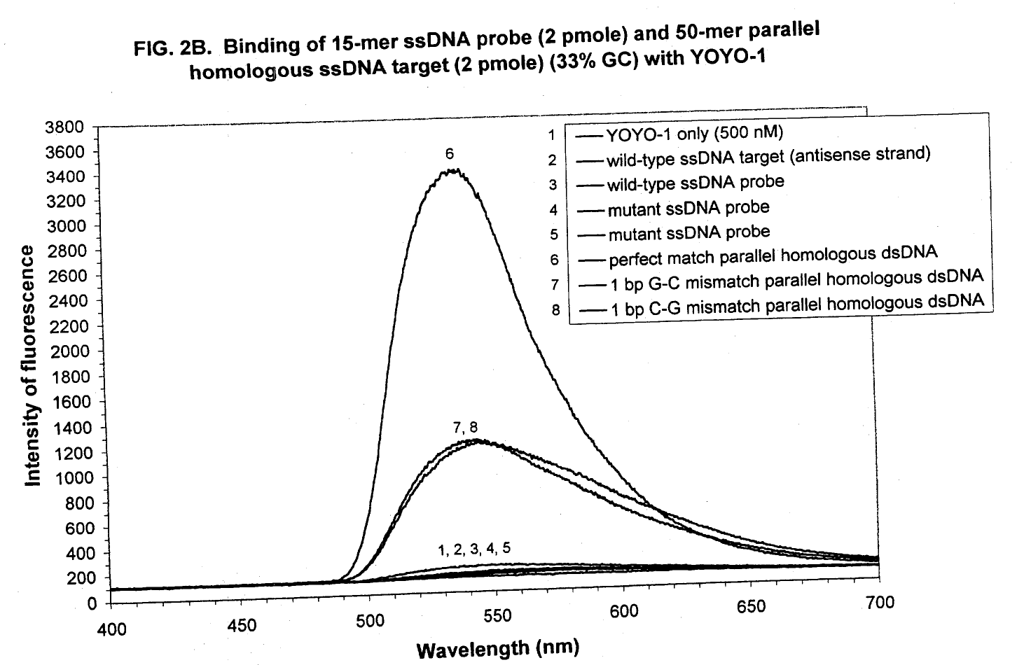

When the ssDNA Probe No. 3 (with a 53% CC content) was reacted with the 50-mer wild-type antisense strand of SEQ ID NO:5 or with the 50-mer mutant antisense strand of SEQ ID NO:10 in the presence of YOYO-1, parallel homologous ssDNA:ssDNA duplexes were formed (FIG. 2A). The fluorescent intensity emitted by the 1 bp A-T mismatched parallel homologous duplex (antisense strand of SEQ ID NO:10+Probe No. 3) was 72% lower than that obtained by the perfectly parallel homologous duplex (antisense strand of SEQ ID NO:5+Probe No. 3) (FIG. 2A). This dramatic decrease in fluorescent emission by the parallel homologous duplex containing a 1 bp A-T, strongly suggested that the Watson-Crick A-T binding was hindered by the spatial and/or charge configuration imposed on the A and T bases when part of parallel homologous strands attempting to achieve stable duplex. Control samples comprising each 50-mer ssDNA target plus 500 nM YOYO-1 exhibited levels of fluorescence which ranged from 96% to 97% lower than that observed with the perfectly matched duplexes (FIG. 2A). The level of fluorescence emitted by the 15-mer ssDNA Probe No. 3 plus 500 nM YOYO-1 was slightly greater than that produced by YOYO-l alone. The shift in fluorescent emission wavelength observed with the ssDNA targets and probe is typical of YOYO-1's emission profile in the presence of ssDNA.

Parallel homologous ssDNA:ssDNA duplexes were also formed when the 50-mer wild-type antisense strand of SEQ ID NO:1 (with a 33% GC content) was reacted with the wild-type ssDNA Probe No. 8 or with the mutant ssDNA Probes No. 9 and 10, in the presence of YOYO-1 (FIG. 2B). The fluorescent intensities emitted by the 1 bp G-C mismatched parallel homologous duplex (antisense strand of SEQ ID NO:1+Probe No. 9) and the 1 bp C-G mismatched parallel homologous duplex (antisense strand of SEQ ID NO:1+Probe No. 10) were 67% and 66% lower, respectively, than that obtained by the perfectly parallel homologous duplex (antisense strand of SEQ ID NO:1+Probe No. 8) (FIG. 2B). The configuration of the interacting bases in the parallel homologous duplexes was unfavorable for Watson-Crick complementary G-C binding, resulting in a decrease in fluorescent emission indicative of a 1 bp mismatch. Control samples consisting of the 50-mer ssDNA target plus 500 nM YOYO-1 or each of the 15-mer ssDNA probes plus 500 nM YOYO-1 resulted in levels of fluorescence that were slightly greater than that produced by YOYO-1 alone (FIG. 2B).

Therefore, the interacting base pairs in parallel homologous dsDNA duplexes, formed in the presence of YOYO-1, adopt a configuration that is unfavorable for binding between Watson-Crick complementary base pairs, resulting in such duplexes appearing to contain 1 bp mismatches.

We are led to envisage how mismatches in binding sequences, whether occurring as part of a hairpin or multistrand complex can cause energetic and repeated motion as the base sequences try to achieve the stability of the ideal binding configuration under either binding motif. It is expected that binding strength of base pairs upstream or downstream of nucleation sites, metal ions and other factors will have a bearing on the attempts to achieve bonding.

### Example 3

This example examines the efficiency of antiparallel homologous ssDNA:ssDNA duplex formation facilitated by YOYO-l or by monovalent cations.

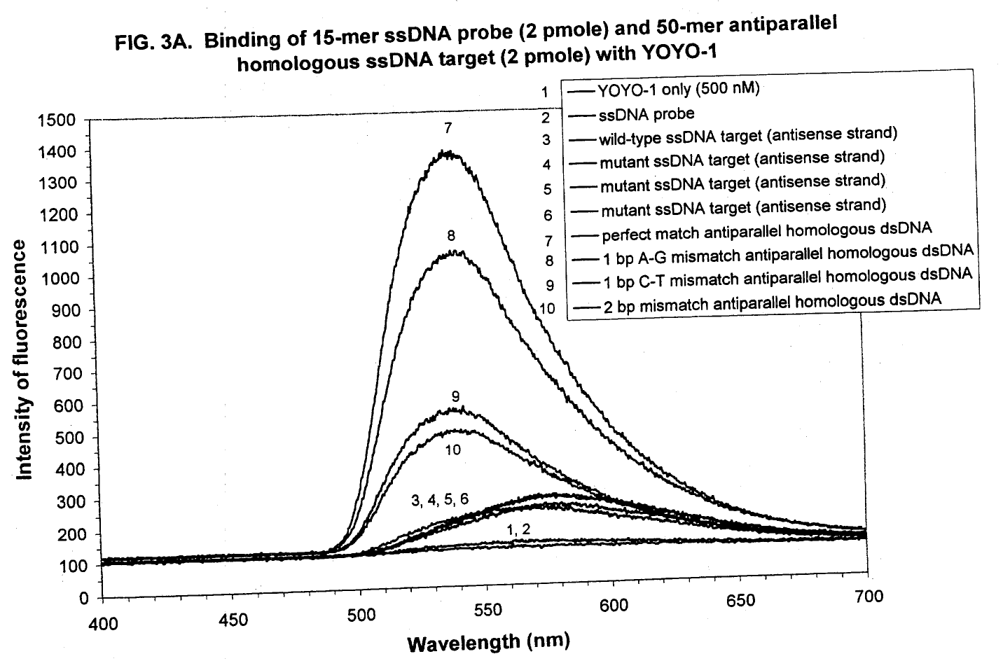

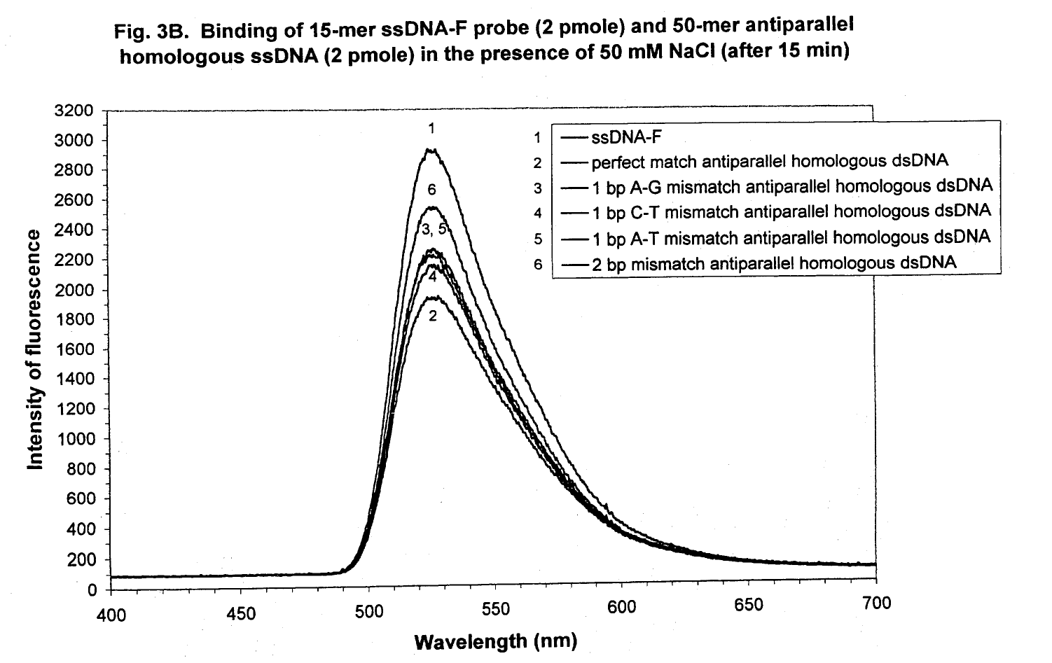

The hybridization reactions, giving rise to the data illustrated in FIG. 3A, each contained the following mixture: 2 pmoles of ssDNA target, 2 pmoles of ssDNA probe, 0.5×TBE and 500 nM of YOYO-1 in a final volume of 40 μl. The reaction mixtures were incubated at room temperature (21° C.) for 5 minutes, placed into a quartz cuvette, irradiated with an argon ion laser beam having a wavelength of 488 nm and monitored for fluorescent emission. The intensity of fluorescent emission was plotted as a function of wavelength for each sample analyzed.

In FIG. 3B, the hybridization reaction mixtures (40 μL) each contained the following: 2 pmoles of ssDNA target, 2 pmoles of 5′-fluorescein labeled ssDNA probe, 10 mM Tris-HC1, pH 7.5, and 50 mM NaCl. The reaction mixtures were incubated at room temperature (21° C.) for various lengths of time ranging from 1 minute to 60 minutes. Following incubation, samples were placed into a quartz cuvette, irradiated with an argon ion laser beam having a wavelength of 488 nm and monitored for fluorescent emission. The intensity of fluorescent emission was plotted as a function of wavelength for each sample analyzed.

Incubation of ssDNA Probe No. 4 with the 50-mer wild-type antisense strand of SEQ ID NO:5 in the presence of YOYO-1 resulted in antiparallel homologous ssDNA:ssDNA complex formation (FIG. 3A). Although the efficiency of antiparallel homologous complex formation was only 65% that of conventional antiparallel complementary dsDNA formation (compare FIGS. 1A and 3A), recognition and binding of antiparallel homologous ssDNA sequences did occur, facilitated by YOYO-L. This result was completely unanticipated. Furthermore, antiparallel homologous ssDNA:ssDNA complexes comprising wild-type sequences were clearly distinguished from those comprising 1 bp or 2 bp mismatches. The fluorescent intensities emitted by the 1 bp A-G mismatched DNA complex (antisense strand of SEQ ID NO:7+Probe No. 4), the 1 bp C-T mismatched DNA complex (antisense strand of SEQ ID NO:6+Probe No. 4), and the consecutive 2 bp mismatched DNA complex (antisense strand of SEQ ID NO:8+Probe No. 4) were 25%, 65% and 71% lower, respectively, than that obtained by the perfect antiparallel homologous complex (antisense strand of SEQ ID NO:5+Probe No. 4) (FIG. 3A). As the degree of homology between the probe and target decreased, the level of fluorescent emission decreased. Control samples comprising each 50-mer ssDNA target plus 500 nM YOYO-1 exhibited levels of fluorescence which ranged from 88% to 90% lower than that observed with the perfectly matched complexes (FIG. 3A). The level of fluorescence emitted by the 15-mer ssDNA Probe No. 4 plus 500 nM YOYO-1 was slightly greater than that produced by YOYO-1 alone.

Antiparallel homologous ssDNA:ssDNA complex formation was further examined using ssDNA targets and ssDNA-F probes both in the presence and absence of 50 mM NaCl. After 15 minutes of incubation of ssDNA-F Probe No. 6 with the 50-mer wild-type antisense strand of SEQ ID NO:5 in the presence of 50 mM NaCl, antiparallel homologous ssDNA:ssDNA-F complexes were formed, as indicated by the 34% decrease in fluorescence observed compared to that emitted by Probe No. 6 alone (FIG. 3B). The efficiency of antiparallel homologous complex formation was 62% that of antiparallel complementary complex formation following a 15 minute incubation (data not shown). By contrast, antiparallel homologous ssDNA:ssDNA-F complexes that contained a 1 bp A-G mismatch (antisense strand of SEQ ID NO:7+Probe No. 6), a 1 bp C-T mismatch (antisense strand of SEQ ID NO:6+Probe No. 6), a 1 bp A-T mismatch (antisense strand of SEQ ID NO:10+Probe No. 6), and a consecutive 2 bp mismatch (antisense strand of SEQ ID NO:8+Probe No. 6), produced a 24%, 26%, 23% and a 13% decrease in fluorescence, respectively, compared to that emitted by Probe No. 6 alone after a 15 minute incubation (FIG. 3B). The configuration of the interacting bases in the antiparallel homologous duplexes was apparently unfavorable for Watson-Crick complementary A-T binding, resulting in a change in fluorescent emission indicative of a 1 bp mismatch. Less antiparallel homologous complex formation occurred following a 30 minute incubation in the presence of 50 mM NaCl (data not shown). No complex formation was evident after 45 minutes of incubation. Similar rates of antiparallel homologous complex formation and stability were observed in Tris buffer without NaCl (data not shown).

Promoted by YOYO-1 or NaCl, recognition and binding of antiparallel homologous ssDNA sequences occurred in a configuration which allowed the discrimination between perfectly matched ssDNA:ssDNA complexes and those containing a 1 bp or 2 bp mismatch. The interaction of the base pairs in the antiparallel homologous duplex resulted in a conventional Watson-Crick A-T base pair being destabilizing as a mismatch.

### Example 4

This example demonstrates the efficiency of parallel complementary ssDNA:ssDNA complex formation promoted by monovalent cations. The hybridization reaction mixtures (40 μl) each contained the following: 2 pmoles of ssDNA target, 2 pmoles of 5′-fluorescein labeled ssDNA probe, 10 mM Tris-HCl, pH 7.5, and 50 mM NaCl. The reaction mixtures were incubated at room temperature (21° C.) for various lengths of time ranging from 1 minute to 60 minutes. Following incubation, samples were placed into a quartz cuvette, irradiated with an argon ion laser beam having a wavelength of 488 nm and monitored for fluorescent emission. The intensity of fluorescent emission was plotted as a function of wavelength for each sample analyzed.

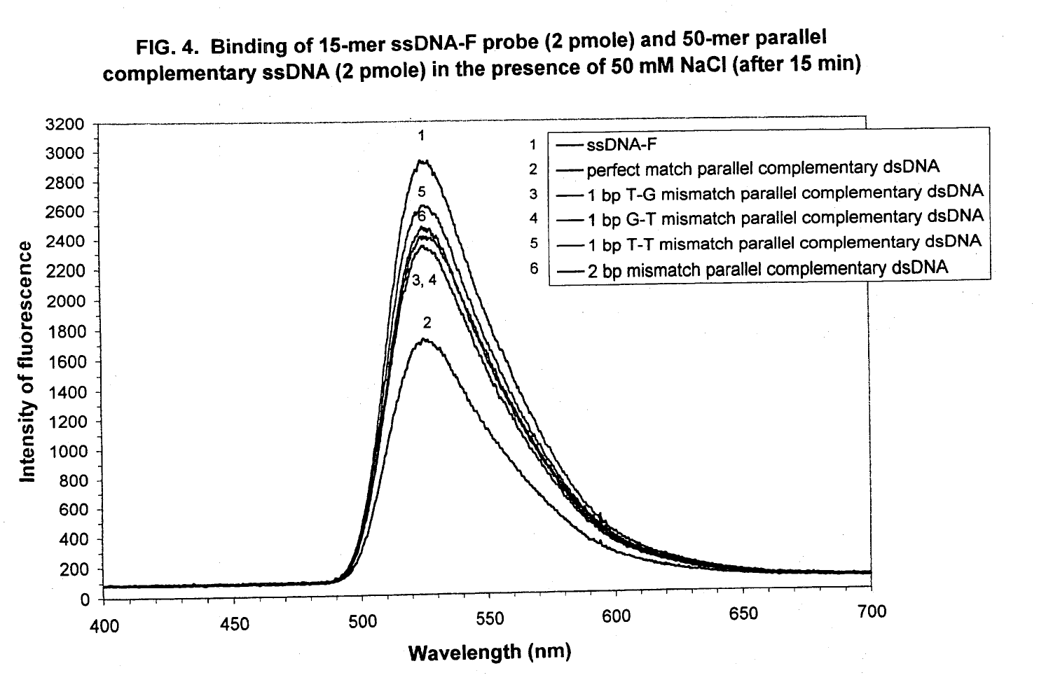

After a 15 minute incubation in the presence of 50 mM NaCl, ssDNA:ssDNA-F duplexes consisting of perfectly complementary sequences (sense strand of SEQ ID NO:5+Probe No. 6) formed readily, resulting in a 41% decrease in fluorescent emission intensity compared to that emitted by Probe No. 6 alone (FIG. 4). This high efficiency of parallel complementary duplex formation was completely unexpected. By contrast, incompletely complementary ssDNA:ssDNA-F complexes containing a 1 bp T-G mismatch (sense strand of SEQ ID NO:7+Probe No. 6), a 1 bp G-T mismatch (sense strand of SEQ ID NO:6+Probe No. 6), a 1 bp T-T mismatch (sense strand of SEQ ID NO:10+Probe No. 6), and a consecutive 2 bp mismatch (sense strand of SEQ ID NO:8+Probe No. 6), generated an 18%, 20%, 10% and 16% decrease, respectively, in fluorescent emission intensity compared to that exhibited by Probe No. 6 alone (FIG. 4).

Once formed in the presence of 50 mM NaCl, the perfectly matched parallel complementary duplexes were very stable, resulting in a 40% and 47% decrease in fluorescent emission after 30 minutes and 45 minutes of incubation, respectively, compared to that emitted by Probe No. 6 alone (data not shown). The 1 bp and 2 bp mismatched parallel complementary complexes were much less stable after 30 minutes and 45 minutes of incubation in the presence of 50 mM NaCl (data not shown). The rate and efficiency of parallel complementary ssDNA:ssDNA-F formation was very similar to that of antiparallel complementary ssDNA:ssDNA-F formation during the first 45 minutes of incubation in the presence of 50 mM NaCl (data not shown). While antiparallel complementary complexes continued to form easily after 60 minutes of incubation in 50 mM NaCl, no parallel complementary complex formation was evident at this time (data not shown).

NaCl facilitated DNA complex formation between a ssDNA-F probe and an antiparallel complementary ssDNA target, or between a ssDNA-F probe and a parallel complementary ssDNA target, with similar efficacy, to allow differentiation between perfectly matched complexes and those containing a 1 bp or 2 bp mismatch.

### Example 5

Examples 1 to 4 demonstrated alternate base recognition and binding motifs occurring between antiparallel or parallel ssDNA probes, and complementary or homologous ssDNA targets to generate ssDNA:ssDNA duplexes, other than the conventional antiparallel Watson-Crick complementary dsDNA complexes. This example will show that bases are capable of recognizing and interacting with both complementary and homologous bases at the same time.

Samples of two pmoles of ssDNA Probe No. 3 were heated at 95° C. for 10 minutes and allowed to cool to room temperature for 30 minutes in the presence of various concentrations of a free base, resulting in ssDNA probes containing conjugated bases. Duplicate samples of ssDNA Probe No. 3 were similarly denatured and cooled in the absence of added free bases to generate non-conjugated ssDNA probes. Two pmoles of these conjugated or non-conjugated ssDNA probes were then mixed with 2 pmoles of ssDNA target in the presence of 500 nM YOYO-L and 0.5×TBE in a final reaction volume of 40 μl. The reaction mixtures were incubated at room temperature (21° C.) for 5 minutes, placed into a quartz cuvette, irradiated with an argon ion laser beam having a wavelength of 488 nm, and monitored for fluorescent emission. The intensity of fluorescence was plotted as a function of wavelength for each sample analyzed.

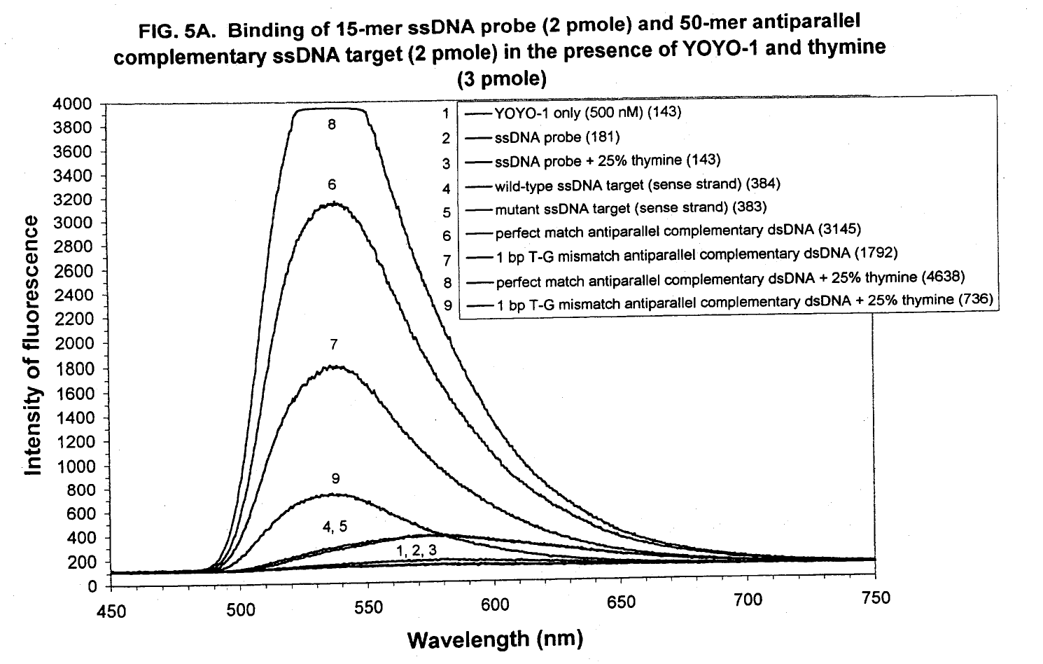

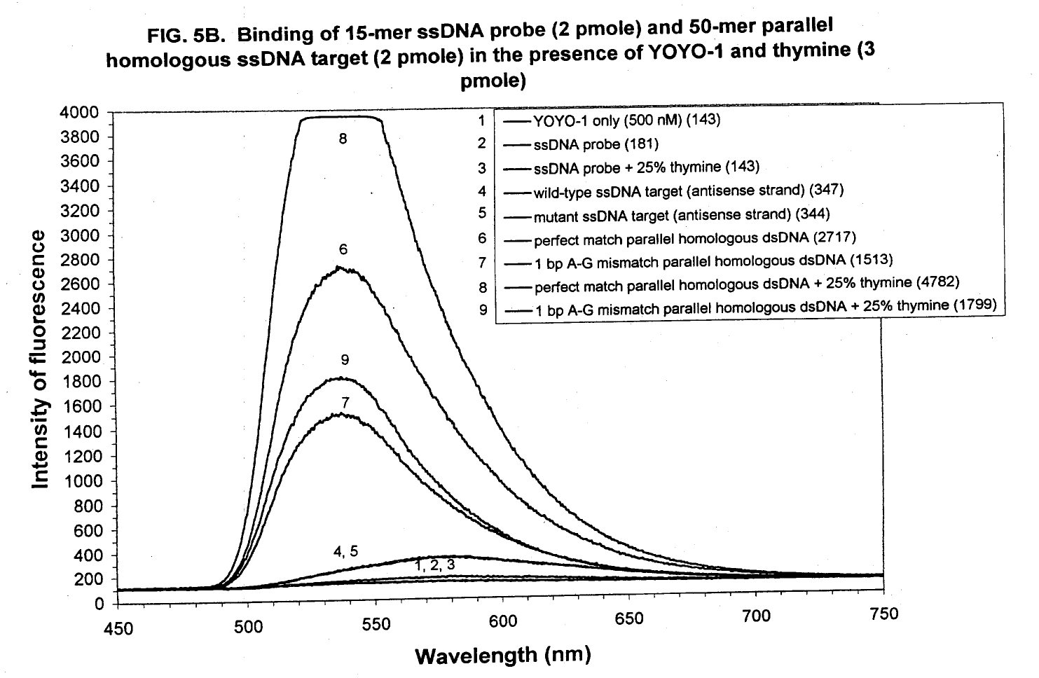

When the non-conjugated ssDNA Probe No. 3 was reacted with the 50-mer wild-type sense strand of SEQ ID NO:5 or with the 50-mer mutant sense strand of SEQ ID NO:7, in the presence of YOYO-1, antiparallel complementary ssDNA:ssDNA complexes were formed (FIG. 5A). The fluorescent intensity emitted by the 1 bp T-G mismatched antiparallel complementary duplex (sense strand of SEQ ID NO:7+Probe No. 3) was 45% lower than that obtained by the perfectly matched antiparallel complementary duplex (sense strand of SEQ ID NO:5+Probe No. 3). Control samples comprising each 50-mer ssDNA target plus 500 nM YOYO-1 exhibited levels of fluorescence which ranged from 92% to 93% lower than that observed with the perfectly matched duplexes (FIG. 5A). The level of fluorescence emitted by the 15-mer ssDNA Probe No. 3 plus 500 nM YOYO-1 was slightly greater than that produced by YOYO-1 alone.

When the ssDNA Probe No. 3 was reacted with the 50-mer wild-type antisense strand of SEQ ID NO:5 in the presence of YOYO-1, the efficiency of parallel homologous ssDNA:ssDNA duplex formation was 14% lower than the efficiency of antiparallel complementary ssDNA:ssDNA duplex formation (compare FIGS. 5A and 5B). The 1 bp A-G mismatched parallel homologous duplex formed when the 50-mer mutant antisense strand of SEQ ID NO:7 was reacted with the ssDNA Probe No. 3 in the presence of YOYO-1, produced a fluorescent emission intensity that was 47% lower than that emitted by the perfectly parallel homologous duplex (FIG. 5B).

The 15-mer ssDNA Probe No. 3 contains six adenine bases. Conjugation of 2 pmoles of ssDNA Probe No. 3 with 3 pmoles of free thymine could result in 25% of the complementary A or 100% of the homologous T within Probe No. 3 bound to the added thymine. Complementary A-T binding is energetically preferred. Reaction of 2 pmoles of ssDNA Probe No. 3 (conjugated with 3 pmoles of thymine) with 2 pmoles of the wild-type antisense strand of SEQ ID NO:5 in the presence of YOYO-1 resulted in dramatically enhanced parallel homologous ssDNA:ssDNA complex formation (FIG. 5B). Twenty-five percent conjugation of the ssDNA probe with 3 pmoles of thymine increased parallel homologous complex formation between the perfectly homologous sequences by 78%. This augmentation of parallel homologous complex formation can be linked to the ability of the adenines in Probe No. 3 to interact simultaneously with the conjugated complementary thymine bases, as well as with the homologous adenines in the ssDNA target. Moreover, interaction with available complementary bases was not deleterious to the homologous binding configuration adopted by the homologous bases and their neighbors.

By contrast, the efficiency of formation of parallel homologous complexes containing a 1 bp A-G mismatch (antisense strand of SEQ ID NO:7+Probe No. 3) were increased by 16% when Probe No. 3 was conjugated 25% with thymine than when non-conjugated Probe No. 3 was used (FIG. 5B). This corresponded to a 65% reduction in fluorescent emission intensity for the 1 bp A-G mismatched parallel homologous complex compared to that observed for the perfectly matched parallel homologous complex when the T-conjugated Probe No. 3 was used. Conjugation of the ssDNA probe increased the specificity in discriminating between perfectly matched parallel homologous complexes and 1 bp mismatched parallel homologous complexes.

Remarkably, perfectly matched antiparallel complementary ssDNA:ssDNA complex formation was enhanced by 48% when Probe No. 3 conjugated 25% with thymine was reacted with the sense strand of SEQ ID NO:5 in the presence of YOYO-1 (FIG. 5A). The simultaneous interaction of an adenine in Probe No. 3 with the conjugated complementary thymine and the complementary T in the ssDNA target augmented formation of the perfectly matched antiparallel complementary complex. Remarkably, formation of the 1 bp T-G mismatched antiparallel complementary complex was very inefficient when T-conjugated Probe No. 3 was used, resulting in an 88% decrease in fluorescent emission intensity compared to that generated by the perfectly matched antiparallel complementary complex containing conjugated T (FIG. 5A). It is also remarkable that discrimination between perfectly matched and 1 bp mismatched antiparallel complementary ssDNA:ssDNA complexes was greatly enhanced by use of conjugated ssDNA probes in the presence of YOYO-1.

Twenty-five percent conjugation of Probe No. 3 with cytosine or guanosine also increased the efficiency of both antiparallel complementary and parallel homologous ssDNA:ssDNA complex formation in the presence of YOYO-1, as well as improved the specificity in differentiation between perfectly matched complexes and 1 bp mismatched complexes (data not shown).

Formation of ssDNA:ssDNA complexes comprising conjugated bases proves that the bases in a sequence can recognize and interact specifically and simultaneously with both complementary and homologous bases provided the conjugated base is a Watson-Crick complement to a base on the strand which binds specifically to another strand. The recognition and binding configurations between bases in a ssDNA probe, conjugated bases and bases in a ssDNA target may be similar to the base configurations formed in antiparallel and parallel dsDNA:ssDNA complexes described herein.

### Example 6

Example 6 demonstrates quadruplex DNA formation between dsDNA targets containing mixed base sequences and homologous dsDNA probes labeled with fluorescein. Quadruplex DNA formation is enhanced by the presence of monovalent cations added to the reaction.

Complementary sense and antisense 15-mer ssDNA sequences were synthesized, purified by HPLC and annealed as above to generate 15-mer dsDNA probes. DsDNA probes were diluted in ddH₂O at a concentration of 1 pmole/μl.

Probe No. 11 was a 15-mer dsDNA probe with an attached fluorescein moiety at each 5′ position, and was designed to be completely homologous to a central 15 bp segment of the 50-mer wild-type target DNA (SEQ ID NO:5).

The sequence for the sense strand of Probe No. 11 (SEQ ID NO:22) was: 5′-Flu-CTG TCA TCT CTG GTG-3′.

The sequence for the antisense strand of Probe No. 11 (SEQ ID NO:22) was: 5′-Flu-CAC CAG AGA TGA CAG-3′.

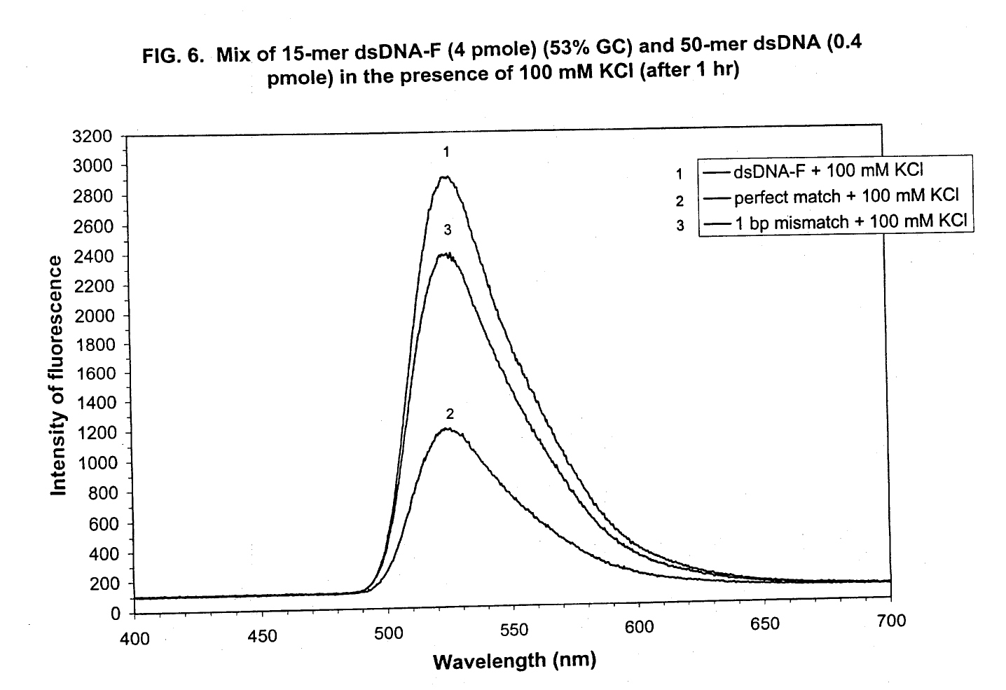

Each hybridization reaction mixture (40 μl) contained the following: 0.4 pmoles of target dsDNA, 4 pmoles of 5′-fluorescein labeled dsDNA Probe No. 11, 10 mM Tris-HCl, pH 7.5 and 100 mM KCl. The reaction mixtures were incubated at room temperature (21° C.) for 1 hour, without prior denaturation. Samples were placed into a quartz cuvette, irradiated with an argon ion laser beam having a wavelength of 488 nm and monitored for fluorescent emission. The maximum fluorescent intensities occurred at a wavelength of 525 nm, the emission wavelength for fluorescein. FIG. 6 shows the intensity of fluorescence plotted as a function of wavelength for each sample analyzed.

In the absence of KCl, no binding between the dsDNA targets and Probe No. 11 was detected, resulting in similar fluorescent intensities observed when wild-type dsDNA target (SEQ ID NO:5) or mutant dsDNA target (SEQ ID NO:7) were mixed with dsDNA Probe No. 11 or when dsDNA Probe No. 11 was present alone (data not shown).

After a 1 hour incubation at 21° C. in the presence of 100 mM KCl, dsDNA:dsDNA-F quadruplexes consisting of perfectly homologous sequences on dsDNA target (SEQ ID NO:5) and dsDNA Probe No. 11 formed readily, resulting in a 62% decrease in the intensity of fluorescent emission compared to that emitted by dsDNA Probe No. 11 alone (labeled dsDNA-F) (FIG. 6). In contrast, incompletely homologous dsDNA:dsDNA-F quadruplexes (SEQ ID NO:7+Probe No. 11), containing a 1 base pair mismatch were less stable in these reaction conditions, yielding only an 18% decrease in fluorescent intensity compared to that exhibited by dsDNA Probe No. 11 alone.

The presence of monovalent cations, such as K⁺, at specific concentrations was sufficient to allow quadruplex formation between dsDNA targets and dsDNA probes labeled with fluorescein in the absence of prior denaturation. Quadruplex formation occurred on the basis of homologous base pair affinities, with a measurable and significantly greater amount of quadruplex formation between fully homologous duplex strands. Moreover, the reaction occurred at room temperature within just 1 hour of incubation at a ratio of probe to target of 10 to 1, using natural dsDNA. The dsDNA targets and dsDNA probe used in this example were homologous, contained 53% GC content, and did not contain homopurine or homopyrimidine stretches on any DNA strand. The assay of the invention was able to identify perfectly homologous dsDNA sequences and those containing a pair of mismatched bases, using a dsDNA probe.

### Example 7

The quadruplex DNA assays performed in Example 6 were facilitated by the addition of monovalent cations in the reaction mixtures. The specificity of the assay was further examined utilizing divalent cations to facilitate quadruplex DNA formation with dsDNA targets and dsDNA-F probes possessing 53% GC content.

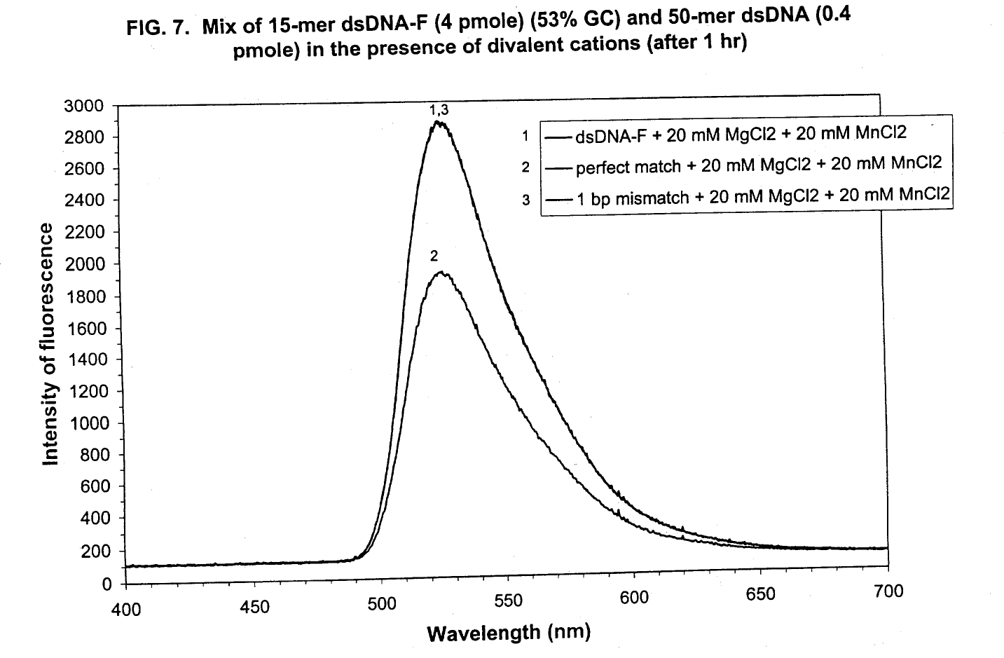

Each hybridization reaction mixture (40 μl) contained the following: 0.4 pmoles of target dsDNA, 4 pmoles of 5′-fluorescein labeled dsDNA Probe No. 11, 10 mM Tris-HCl, pH 7.5, 20 mM MnCl₂ and 20 mM MgCl₂. The reaction mixtures were incubated at room temperature (21° C.) for 1 hour, without prior denaturation. Samples were placed into a quartz cuvette, irradiated with an argon ion laser beam having a wavelength of 488 nm and monitored for fluorescent emission. FIG. 7 shows the intensity of fluorescence plotted as a function of wavelength for each sample analyzed.

When dsDNA-F Probe No. 11 (with a 53% GC content) was incubated with the 50-mer wild-type dsDNA target (SEQ ID NO:5) or the mutant dsDNA target (SEQ ID NO:7) in the presence of 20 mM MnCl₂ and 20 mM MgCl₂, quadruplexes were formed at room temperature under non-denaturing conditions. While perfectly homologous DNA quadruplexes yielded the maximum decrease in fluorescent intensity, a 34% decrease, the less favourable dsDNA:dsDNA-F quadruplexes containing a 1 bp mismatch (SEQ ID NO:7+Probe No. 11) produced a fluorescent intensity that was about the same as that observed with dsDNA Probe No. 11 alone (FIG. 7).

The presence of divalent cations such as Mn⁺² and Mg⁺² facilitated quadruplex formation under non-denaturing conditions to allow accurate discrimination between fully homologous dsDNA target and dsDNA probe quadruplexes, and quadruplex sequences containing a pair of bases which are mismatched.

### Example 8

The quadruplex DNA assays performed in Examples 6 and 7 were facilitated by the addition of either monovalent cations or divalent cations in the reaction mixtures. The next Example demonstrates the specificity of the homologous quadruplex DNA assay when the DNA intercalator, YOYO-1, is employed.

Complementary sense and antisense 15-mer ssDNA sequences were synthesized, purified by HPLC and annealed as above to generate 15-mer dsDNA probes. DsDNA probes were diluted in ddH₂O at a concentration of 1 pmole/μl.

Probe No. 12 was a 15-mer dsDNA probe identical in sequence to Probe No. 11, but without the attached 5′ fluorescein moieties.

The sequence for the sense strand of Probe No. 12 (SEQ ID NO:23) was: 5′-CTG TCA TCT CTG GTG-3′.

The sequence for the antisense strand of Probe No. 12 (SEQ ID NO:23) was: 5′-CAC CAG AGA TGA CAG-3′.

Each hybridization reaction mixture (40 μl) contained the following: 0.4 pmoles of dsDNA target, 4 pmoles of dsDNA Probe No. 12, 0.5×TBE and 100 nM of YOYO-1. The reaction mixtures were incubated at 21° C. for 5 minutes, placed into a quartz cuvette, irradiated with an argon ion laser beam having a wavelength of 488 nm and monitored for fluorescent emission. The intensity of fluorescent emission was plotted as a function of wavelength for each sample analyzed.

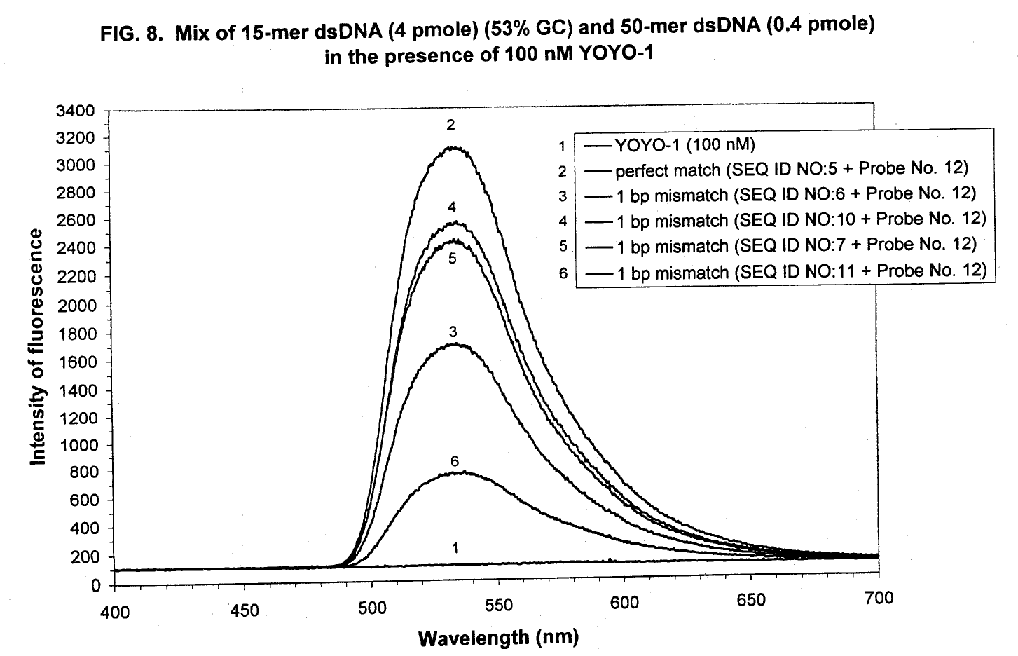

The fluorescent intensities observed when no target or probe was present (YOYO-1 only) are shown in FIG. 8. FIG. 8 also shows the fluorescent intensities observed when the reaction mixtures combined dsDNA Probe No. 12 with wild-type 50-mer dsDNA target (SEQ ID NO:5) which contained homologous sequences, or with four other dsDNA targets which, but for one mismatched pair of bases, contained sequences which were homologous to the base sequences in the dsDNA Probe No. 12. Homologous wild-type dsDNA target (SEQ ID NO:5) when present in the reaction mixture with the dsDNA Probe No. 12 produced the greatest fluorescent intensity. Mismatched dsDNA targets when incubated with dsDNA Probe No. 12 in the reaction mixture yielded lesser fluorescent intensity values ranging from 20% less for dsDNA target (SEQ ID NO:10) to 80% less for dsDNA target (SEQ ID NO:11), compared to that achieved by perfectly matched quadruplexes (FIG. 8).

It was observed that homologous quadruplexes, stabilized by YOYO-1 intercalation, formed more readily between a dsDNA target and a dsDNA probe when that probe contained perfectly homologous sequences, than when there was a single pair of bases which were not homologous, that is to say identical, to a pair of bases in the dsDNA target. The quadruplex complexes described in the foregoing three examples are referred to by us as mirror homologous.

### Example 9

In this example, 50-mer dsDNA targets were exposed to a 53% GC 15-mer dsDNA probe (Probe No. 13), wherein Watson-Crick complementarity exists between bases of the strands of the probe and proximal bases of the strands of the target when the major groove of one duplex is placed in the minor groove of the other duplex. The sequences of bases in the duplex probe are not homologous but are inverted in relation to those in the duplex target. The duplexes, when nested major groove into minor groove, are parallel to one another, and referred to by us as nested complementary.

Complementary sense and antisense 15-mer ssDNA sequences were synthesized, purified by HPLC and annealed as above to generate 15-mer dsDNA probes. DsDNA probes were diluted in ddH₂O at a concentration of 1 pmole/μl.

The sequence for the sense strand of Probe No. 13 (SEQ ID NO:24) was: 5′-GAC AGT AGA GAC CAC-3′.

The sequence for the antisense strand of Probe No. 13 (SEQ ID NO:24) was: 5′-GTG GTC TCT ACT GTC-3′.

Each hybridization reaction mixture (40 μl) contained the following: 0.4 pmoles of target dsDNA, 4 pmoles of dsDNA Probe No. 13, 0.5×TBE and 100 nM of YOYO-1. The reaction mixtures were incubated at room temperature (21° C.) for 5 minutes, placed in a quartz cuvette, irradiated with an argon ion laser beam having a wavelength of 488 nm and monitored for fluorescent emission. The intensity of fluorescent emission was plotted as a function of wavelength for each sample analyzed.

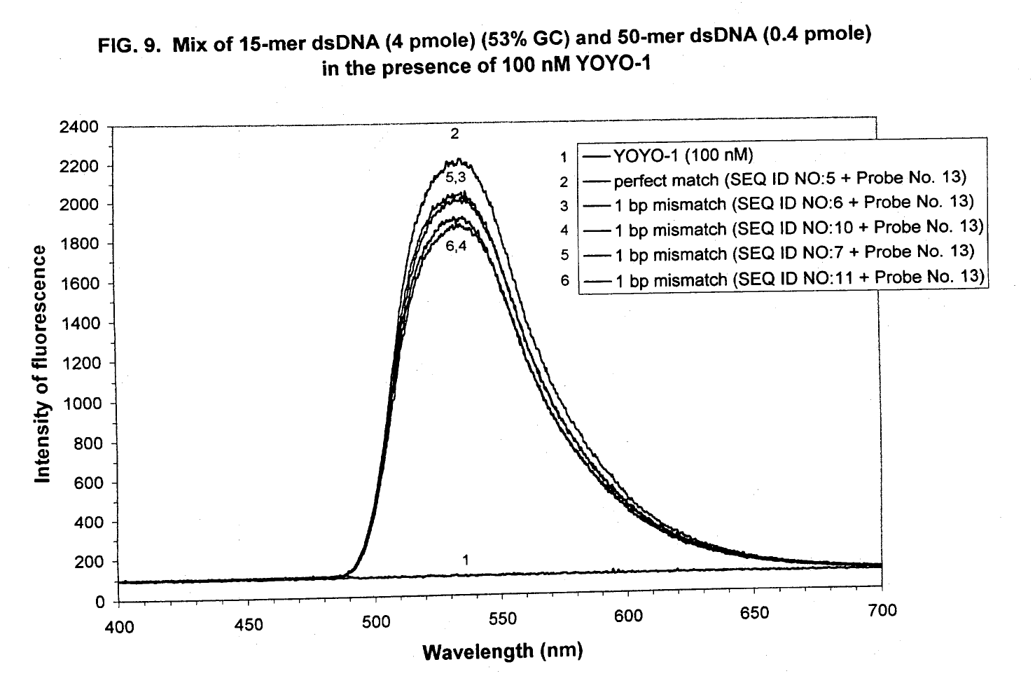

FIG. 9 illustrates that in the absence of prior denaturation, the highest fluorescent intensities were achieved when the wild-type 50-mer dsDNA target (SEQ ID NO:5) was reacted with the 15-mer dsDNA Probe No. 13, which was a perfect match on a nested complementary basis to the dsDNA target (SEQ ID NO:5). The fluorescent intensity is indicative of DNA binding taking place, in this case quadruplex formation between the dsDNA target and the nested complementary dsDNA probe.

Mutant dsDNA targets which were mismatched with the duplex probe by a single pair of bases when matching was assessed on the inverted homology basis of nested complementarity, formed measurably fewer quadruplex complexes with the dsDNA probe, than did the fully complementary wild-type dsDNA target. The various mismatches, which were assayed on a mirror homologous basis in Example 8 were assayed on a nested complementary basis in this example.

As shown in FIG. 9, the fluorescent intensities produced by the quadruplexes formed with the 1 bp mismatched dsDNA targets plus dsDNA Probe No. 13, ranged from 8% to 16% less than that achieved by perfectly matched quadruplexes (SEQ ID NO:5+Probe No. 13).

Greater discrimination in fluorescence was observed between perfectly homologous and partially homologous quadruplexes in Example 8. This suggests that fully complementary or 1 base pair mismatched nested complementary dsDNA probes bind less discriminately to dsDNA targets than do mirror homologous dsDNA probes, which bind with greater specificity.

This example shows that Watson-Crick quadruplex binding between nested complementary DNA duplexes readily occurs in the presence of YOYO-1.

### Example 10

Example 10 demonstrates that the assay of the invention can discriminate between perfectly matched, Watson-Crick complementary dsDNA:ssPNA complexes and dsDNA:ssPNA complexes containing 1 bp, 2 bp and 3 bp mismatches when a cationic decondensing agent, such as the DNA intercalator, YOYO-1 is present.

Each hybridization reaction mixture (40 μl) contained the following: 2 pmoles of target dsDNA, 2 pmoles of ssPNA probe, 0.5×TBE and 500 nM of YOYO-1. The reaction mixtures were incubated at room temperature (21° C.) for 5 minutes, placed into a quartz cuvette, irradiated with an argon ion laser beam having a wavelength of 488 nm and monitored for fluorescent emission. The intensity of fluorescence was plotted as a function of wavelength for each sample analyzed.

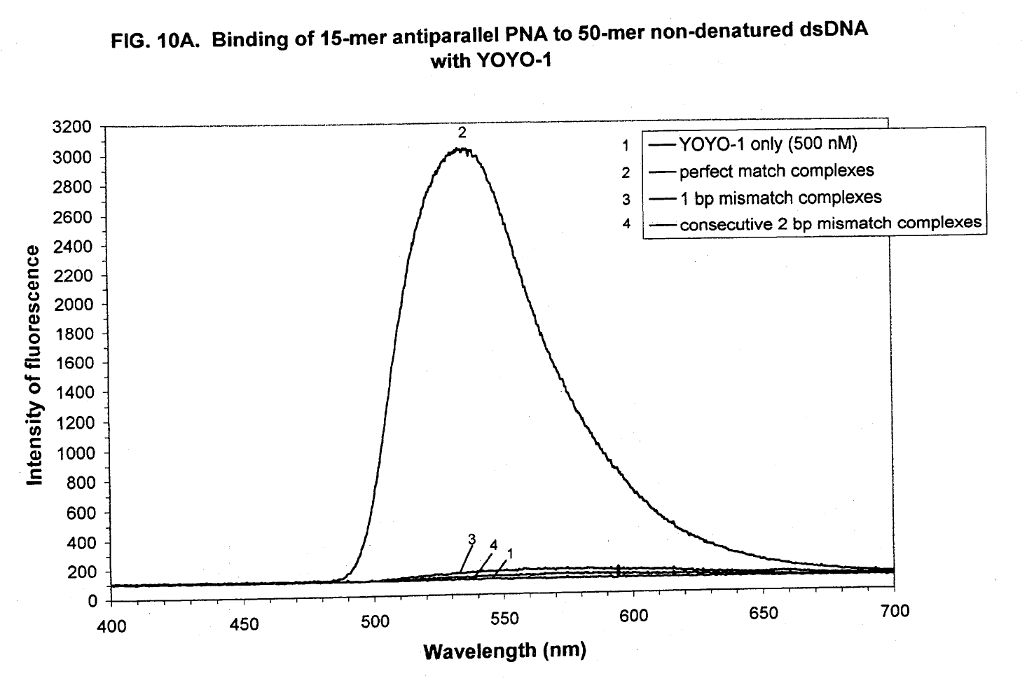

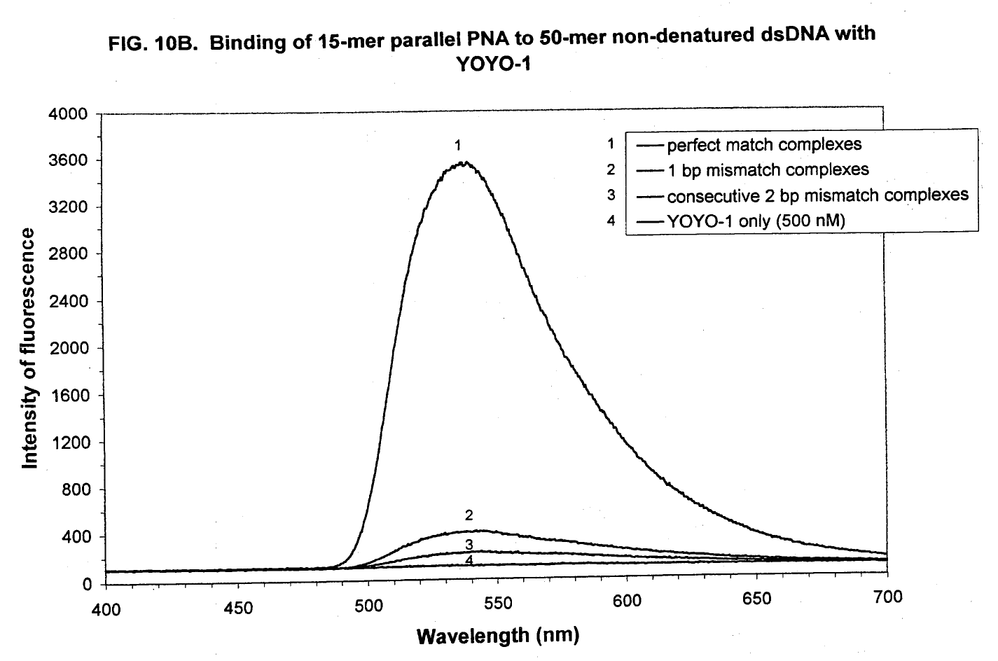

The fluorescent intensities observed when no DNA or PNA was present (YOYO-1 only), or when wild-type SEQ ID NO:1, mutant SEQ ID NO:2 or mutant SEQ ID NO:3 were reacted with antiparallel PNA Probe No. 1 or parallel PNA Probe No. 2 are shown in FIGS. 10A and 10B, respectively. DsDNA:ssPNA complexes consisting of perfectly complementary sequences (SEQ ID NO:1+Probe No. 1) allowed maximum interaction between YOYO-1 and the complexes, yielding the highest fluorescent intensities (FIG. 10A). The fluorescent intensities for a one base pair mismatched dsDNA:ssPNA complex (SEQ ID NO:2+Probe No. 1) and a two base pair mismatched dsDNA:ssPNA complex (SEQ ID NO:3+Probe No. 1) was 97% and 99% lower, respectively, than the perfectly matched dsDNA:ssPNA complex (FIG. 10A). Similarly, when parallel PNA Probe No. 2 was bound to the target dsDNA sequences, the one and two base pair mismatched dsDNA:ssPNA complexes exhibited fluorescent intensities that were 92% and 97% lower, respectively, than the perfectly complementary dsDNA:ssPNA complexes (SEQ ID NO:1+Probe No. 2) (FIG. 10B). Three base pair mismatched dsDNA:ssPNA complexes consisting of SEQ ID NO:4 and Probe No. 1, or SEQ ID NO:4 and Probe No. 2 produced fluorescent intensities that were 99% and 97% lower, respectively, than the perfectly matched dsDNA:ssPNA complexes (data not shown). Control samples comprising 50-mer dsDNA targets plus 500 nM YOYO-1 exhibited levels of fluorescence which were at or below the level of fluorescence observed with 3 bp mismatched complexes (data not shown). The level of fluorescence emitted by either ssPNA probe plus 500 nM YOYO-1 together was identical to that emitted by YOYO-1 alone (data not shown). As the degree of mismatch between the probe and the target increased, the level of interaction of YOYO-1 with the mismatched complexes diminished. Hence the intensity of fluorescent emission decreased. This relationship held whether or not an antiparallel or parallel PNA probe was used. The characteristic level of fluorescence emitted by each complex was monitored over time and was stable between 5 minutes and 24 hours.

Interestingly, when 15-mer target dsDNA sequences were reacted with 15-mer PNA probe sequences, larger differences in fluorescent emission were observed between perfectly matched complexes and 1 or 2 bp mismatched complexes when parallel PNA probes were used, than when antiparallel PNA probes were used (data not shown).

Therefore, the fluorescent intensity assay measuring dsDNA:ssPNA complex formation is able to distinguish between wild-type sequences and those containing 1 bp, 2 bp or 3 bp mutations, without prior denaturation of the duplex DNA target.

### Example 11

The specificity of the assay measuring triplex formation promoted by YOYO-1 was further investigated by reacting wild-type and mutant dsDNA targets of mixed base sequence with antiparallel and parallel ssDNA probes in the absence of prior denaturation of dsDNA targets.

Each hybridization reaction mixture (40 μl) contained the following: 2 pmoles of target dsDNA, 2 pmoles of ssDNA probe, 0.5×TBE and 500 nM of YOYO-1. The reaction mixtures were incubated at room temperature (21° C.) for 5 minutes, placed into a quartz cuvette, irradiated with an argon ion laser beam having a wavelength of 488 nm and monitored for fluorescent emission. The intensity of fluorescence was plotted as a function of wavelength for each sample analyzed.

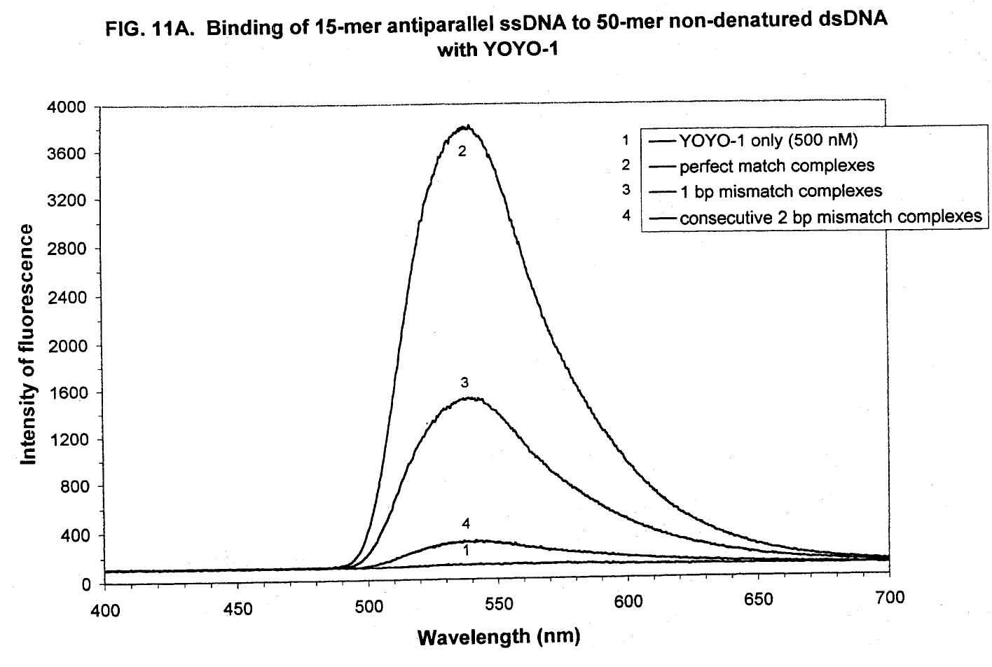

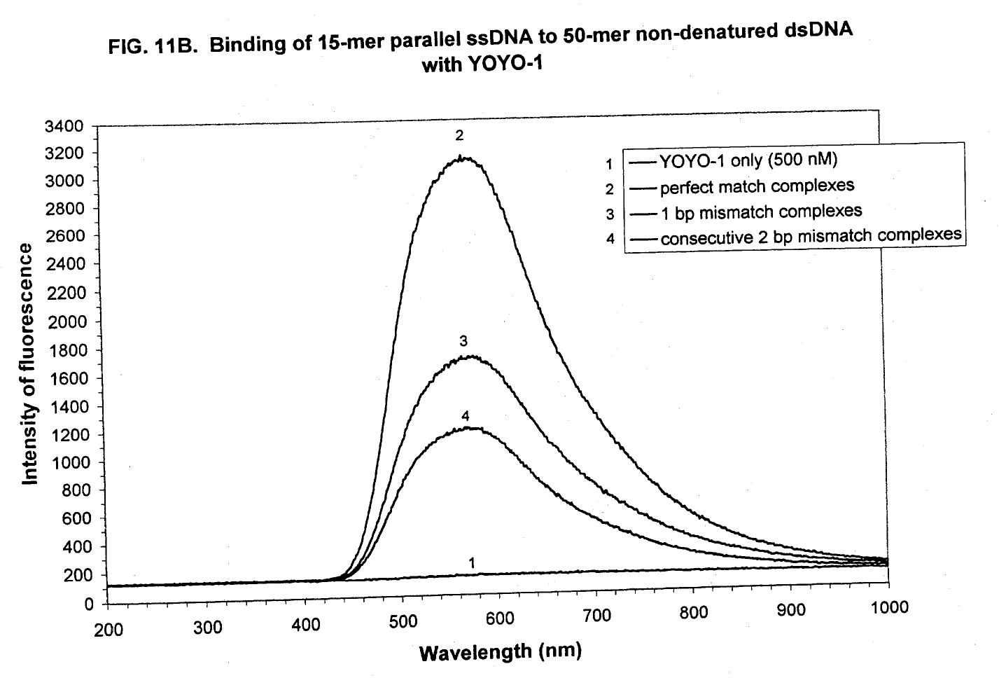

When the antiparallel ssDNA Probe No. 3 (with a 53% GC content) was reacted with the 50-mer wild-type dsDNA target (SEQ ID NO:5) and mutant dsDNA targets (SEQ ID NO:6 and SEQ ID NO:8), dsDNA:ssDNA complexes were formed at room temperature under non-denaturing conditions (FIG. 11A). While perfectly matched DNA complexes emitted the highest fluorescent intensities, incompletely complementary complexes with a 1 bp mismatch (SEQ ID NO:6+Probe No. 3) and a consecutive 2 bp mismatch (SEQ ID NO:8+Probe No. 3) produced fluorescent intensities that were 63% and 95% lower, respectively, than that observed with the perfectly matched sequences (FIG. 11A). The level of fluorescence diminished as the degree of mismatch between the probe and target increased. The characteristic fluorescent intensity exhibited by each complex was monitored over time and was stable between 5 minutes and 24 hours. Control samples comprising 50-mer dsDNA targets plus 500 nM YOYO-1 exhibited levels of fluorescence which were below the level of fluorescence observed with 2 bp mismatched DNA complexes (data not shown). The level of fluorescence generated by the ssDNA probe plus 500 nM YOYO-1 was identical to that achieved by YOYO-1 alone (data not shown). Very similar results were obtained when 15-mer antiparallel ssDNA probes were reacted with wild-type or mutant 50-mer dsDNA targets having 33% GC and 73% GC contents under the same reaction conditions, demonstrating the reliability of the dsDNA:ssDNA complex formation assay utilizing antiparallel ssDNA probes, independent of the percent GC content of the ssDNA probes and dsDNA targets (data not shown).

Similarly, in the presence of YOYO-1, dsDNA:ssDNA complexes were formed when the parallel ssDNA Probe No. 4 was reacted with the 50-mer wild-type dsDNA target (SEQ ID NO:5) and mutant dsDNA targets (SEQ ID NO:6 and SEQ ID NO:8). The fluorescent intensities for a 1 bp mismatched DNA complex (SEQ ID NO:6+Probe No. 4) and a consecutive 2 bp mismatched DNA complex (SEQ ID NO:8+Probe No. 4) were 48% and 65% lower, respectively, than that obtained by the perfectly matched sequences (FIG. 11B). As the degree of mismatch between the probe and target increased, the level of fluorescent emission decreased. Slightly less discrimination between perfectly matched and mismatched DNA complexes was observed when a parallel ssDNA probe was used than when an antiparallel ssDNA probe was used to generate the dsDNA:ssDNA complexes.

YOYO-1 facilitated DNA complex formation between an antiparallel ssDNA probe and dsDNA targets, and between a parallel ssDNA probe and dsDNA targets, to allow differentiation between perfectly matched complexes and those containing 1 bp or 2 bp mismatches, without the requirement for prior denaturation of dsDNA targets.

### Example 12

The complexes formed in Examples 10 and 11 were stabilized by the DNA intercalator, YOYO-1 present in the reaction mixtures. The specificity of the assay was further examined utilizing divalent cations to promote and stabilize complex formation with dsDNA targets and ssDNA-F probes.

Each hybridization reaction mixture (40 μl) contained the following: 0.4 pmoles of target dsDNA, 4 pmoles of 5′-fluorescein labeled ssDNA probe, 10 mM Tris-HCl, pH 7.5, and 1 mM to 20 mM each of MgCl₂ and MnCl₂. The reaction mixtures were incubated at room temperature (21° C.) for various lengths of time ranging from 1 minute to 2 hours, without prior denaturation of dsDNA targets. Following incubation, samples were placed into a quartz cuvette, irradiated with an argon ion laser beam having a wavelength of 488 nm and monitored for fluorescent emission. The maximum fluorescent intensities occurred at a wavelength of 525 nm, the emission wavelength for fluorescein. The intensity of fluorescent emission was plotted as a function of wavelength for each sample analyzed.

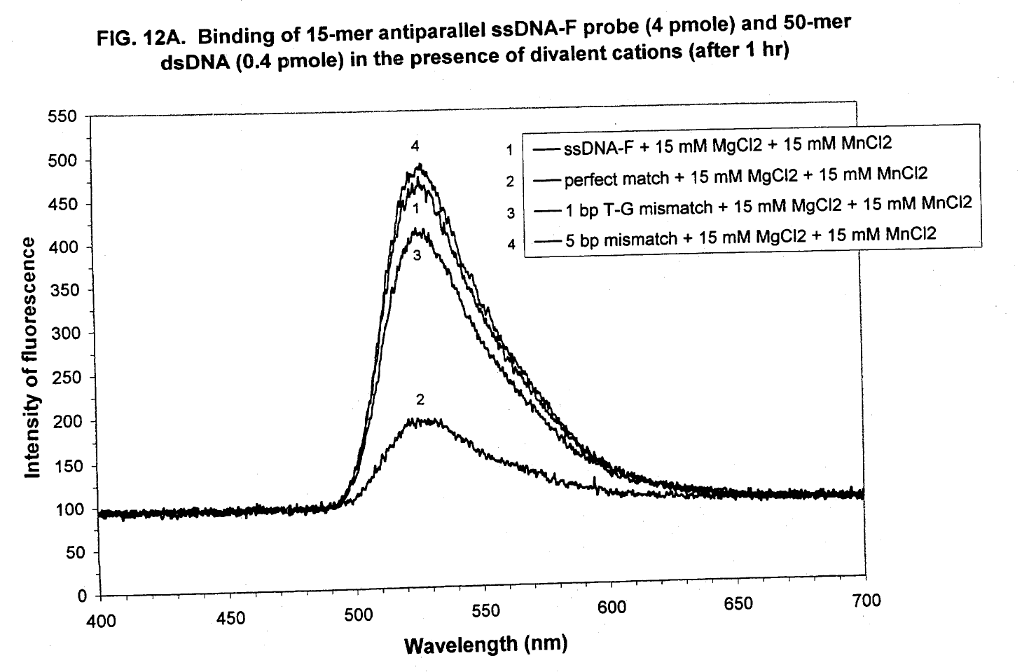

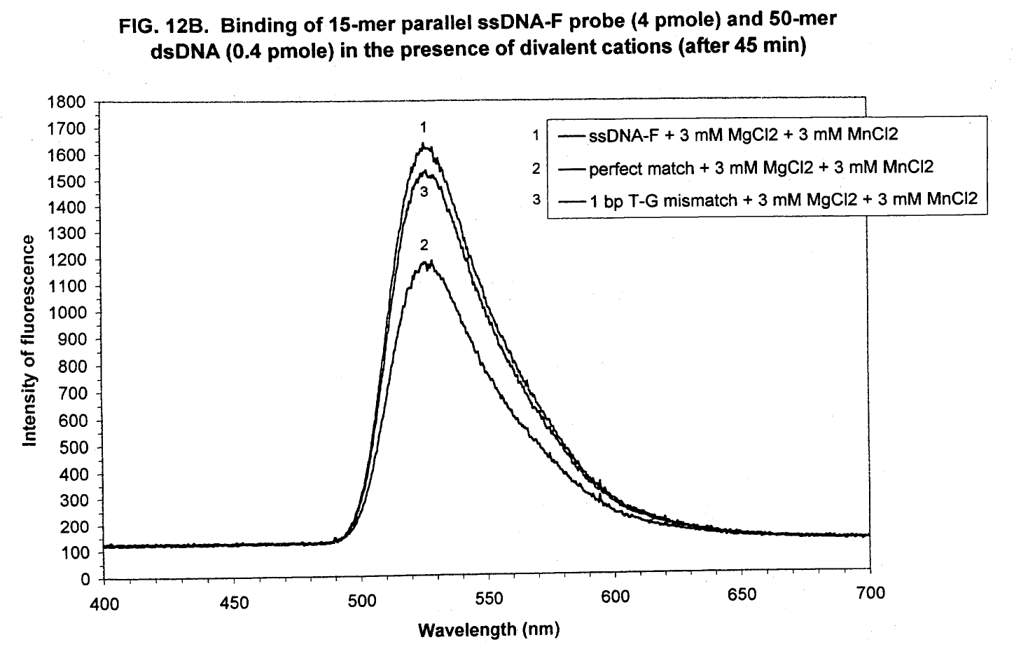

When the antiparallel ssDNA-F Probe No. 5 was incubated for 1 hour with the 50-mer wild-type dsDNA target (SEQ ID NO:5) in the presence of 15 mM MgCl₂ and 15 mM MnCl₂, perfectly complementary dsDNA:ssDNA-F complexes were formed very efficiently, generating a 74% decrease in fluorescence compared to that achieved by Probe No. 5 alone (FIG. 12A). By contrast, dsDNA:ssDNA-F complexes that contained a 1 bp T-G mismatch (SEQ ID NO:7+Probe No. 5) were much less stable in the presence of 15 mM MgCl₂ and 15 mM MnCl₂, yielding a 15% decrease in fluorescence compared to that emitted by Probe No. 5 alone after a 1 hour incubation (FIG. 12A). When Probe No. 5 (containing a 53% GC content) was reacted with the dsDNA target SEQ ID NO:9 (containing a 33% GC content), a 3% increase in fluorescence was observed compared to that obtained by Probe No. 5 alone (FIG. 12A), indicative of no DNA complex formation. This result was expected considering this probe and target combination would result in a 5 bp mismatch.

In the presence of 10 mM MgCl₂ and 10 mM MnCl₂, the dsDNA:ssDNA-F complexes possessing a 53% GC content and containing perfectly complementary sequences (SEQ ID NO:5+Probe No. 5) or a 1 bp T-G mismatch (SEQ ID NO:7+Probe No. 5) generated fluorescent intensities that were 68% and 20% lower, respectively, after an 1 hour incubation, and 76% and 16% lower, respectively, after a 30 minute incubation, than that emitted by Probe No. 5 alone (data not shown). The addition of 5 mM MgCl₂ and 5 mM MnCl₂ (or lower concentrations) was insufficient to allow complex formation between the antiparallel ssDNA-F Probe No. 5 and all dsDNA targets tested following a 1 hour incubation (data not shown).

DsDNA:ssDNA complexes were also formed when the parallel ssDNA Probe No. 6 was reacted with the 50-mer wild-type dsDNA target (SEQ ID NO:5) and mutant dsDNA target (SEQ ID NO:7) In this case DNA complex formation was promoted with much lower concentrations of MgCl₂ and MnCl₂ (i.e. 1-5 mM each) requiring shorter incubation periods. Incubation in the presence of 1 mM MgCl₂ and 1 mM MnCl₂, or 2 mM MgCl₂ and 2 mM MnCl₂ for 15 minutes was sufficient to generate DNA complexes (data not shown). The fluorescent intensities for a perfectly matched DNA complex (SEQ ID NO:5+Probe No. 6) and a 1 bp mismatched DNA complex (SEQ ID NO:7+Probe No. 6) were 29% and 6% lower, respectively, than that obtained by parallel ssDNA Probe No. 6 alone in the presence of 3 mM MgCl₂ and 3 mM MnCl₂ after a 45 minute incubation (FIG. 12B).

Although DNA complexes formed readily at 10 mM MgCl₂ and 10 mM MnCl₂ after a 1 hour incubation, no discrimination between perfectly matched and mismatched complexes was observed when a parallel ssDNA probe was used. Concentrations above 15 mM MgCl₂ and 15 mM MnCl₂ were inhibitory for DNA complex formation with a parallel ssDNA probe (data not shown).

The addition of salt bridging, condensing agents such as divalent cations promoted DNA complex formation between non-denatured dsDNA targets and fluorescently-labeled antiparallel or parallel ssDNA probes, to allow accurate and reliable discrimination between perfectly complementary sequences and those containing 1 bp mutations. The reactions occurred at room temperature within 15-60 minutes of incubation at a ratio of probe to target of 10 to 1. The dsDNA targets and ssDNA probes did not contain homopurine or homopyrimidine stretches of DNA. Despite the presence of 5 pyrimidine bases interspersed within the 15 nucleotide ssDNA probes, DNA complexes formed readily in a sequence specific manner.

### Example 13

The utility of probes of varying directionality was also evaluated when monovalent cations were employed to promote and stabilize complex formation with dsDNA targets.

Each hybridization reaction mixture (40 μl) contained the following: 0.4 pmoles of target dsDNA, 4 pmoles of 5′-fluorescein labeled ssDNA probe, 10 mM Tris-HCl, pH 7.5, and 10 mM to 150 mM NaCl. The reaction mixtures were incubated at room temperature (21° C.) for various lengths of time ranging from 1 minute to 2 hours, without prior denaturation of dsDNA targets. Following incubation, samples were placed into a quartz cuvette, irradiated with an argon ion laser beam having a wavelength of 488 nm and monitored for fluorescent emission. The maximum fluorescent intensities occurred at a wavelength of 525 nm, the emission wavelength for fluorescein. The intensity of fluorescent emission was plotted as a function of wavelength for each sample analyzed.

In the absence of NaCl or presence of 10 mM or 25 mM NaCl, no binding between the dsDNA targets (SEQ ID NO:1 or SEQ ID NO:2) and the antiparallel ssDNA-F Probe No. 7 was detected, after all incubation periods (data not shown).

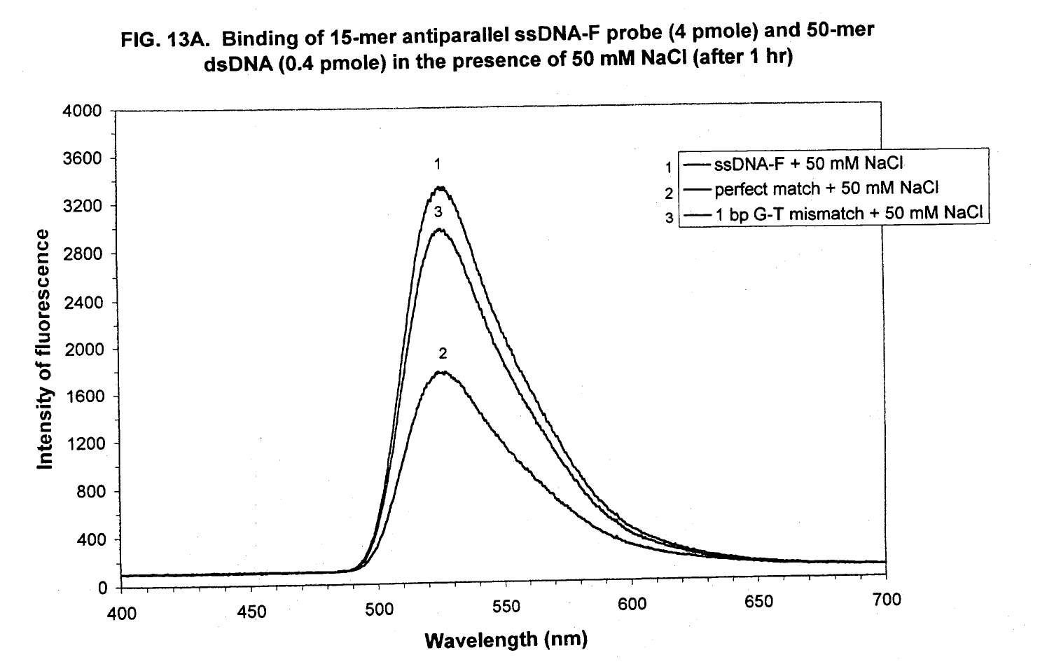

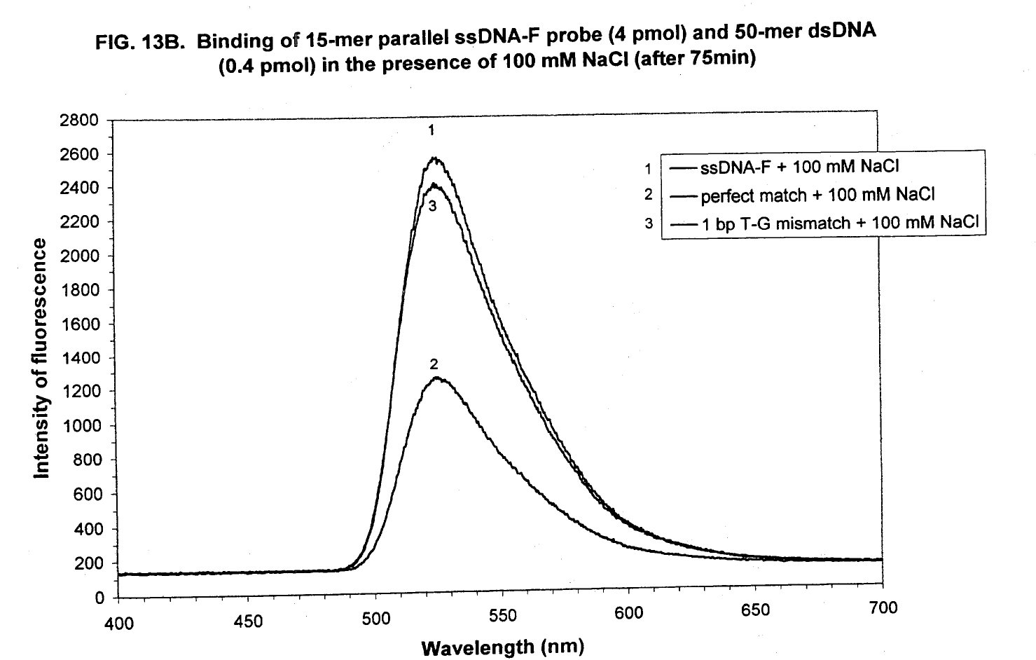

After a 1 hour incubation in the presence of 50 mM NaCl, dsDNA:ssDNA-F complexes consisting of perfectly complementary sequences (SEQ ID NO:1+Probe No. 7) formed readily, resulting in a 49% decrease in fluorescent emission intensity compared to that emitted by the control Probe No. 7, which was similarly incubated in the reaction mixture (FIG. 13A). By contrast, incompletely complementary dsDNA:ssDNA-F complexes containing a 1 bp G-T mismatch (SEQ ID NO:2+Probe No. 7) yielded a 11% decrease in fluorescent emission intensity compared to that exhibited by the Probe No. 7 control sample.

The presence of 75 mM, 100 mM and 125 mM NaCl in the reaction mixture also resulted in fluorescent emission quenching consistent with significant amounts of complex formation between the perfectly matched SEQ ID NO:1 target and antiparallel Probe No. 7, and significantly less quenching when the 1 bp G-T mismatched SEQ ID NO:2 target and Probe No. 7 were present, producing similar fluorescent intensities to that observed in the presence of 50 mM NaCl (data not shown).

DsDNA:ssDNA complexes were also formed when the parallel ssDNA Probe No. 6 was reacted with the 50-mer wild-type dsDNA target (SEQ ID NO:5) and mutant dsDNA target (SEQ ID NO:7) in the presence of 50 mM, 75 mM, 100 mM or 150 mM NaCl. Optimum results were obtained in the presence of 100 mM NaCT. After a 75 minute incubation at room temperature in a reaction mixture containing 100 mM NaCl, the fluorescent emission intensities for a perfectly matched DNA complex (SEQ ID NO:5+Probe No. 6) and a 1 bp mismatched DNA complex (SEQ ID NO:7+Probe No. 6) were 53% and 9% lower, respectively, than that obtained by the control parallel ssDNA Probe No. 6 reacted under the same conditions (FIG. 13B). 50 mM NaCl promoted maximum discrimination between perfectly matched and mismatched complexes in an incubation period of 45 minutes (data not shown). In general, complexes containing either antiparallel or parallel ssDNA probes seemed to form with similar efficiencies at similar NaCl concentrations and incubation periods.

Use of monovalent cations, which are known DNA condensing agents, facilitated DNA complex formation between non-denatured dsDNA targets and fluorescently-labeled antiparallel or parallel ssDNA probes, to allow reliable differentiation between complexes containing perfectly complementary sequences and those containing 1 bp mismatches.

### Example 14

DsDNA:ssDNA complexes facilitated by YOYO-1 readily form at room temperature within 5 minutes of incubation and generate fluorescent emissions at the same level of intensity for hours. Complexes containing base pair mismatches similarly emit fluorescent signals which persist, indicating the same level of complex formation over time. To examine the rate of formation, stability and rate of disassociation of dsDNA:ssDNA complexes formed in the presence of condensing agents such as cations, time course experiments were performed.

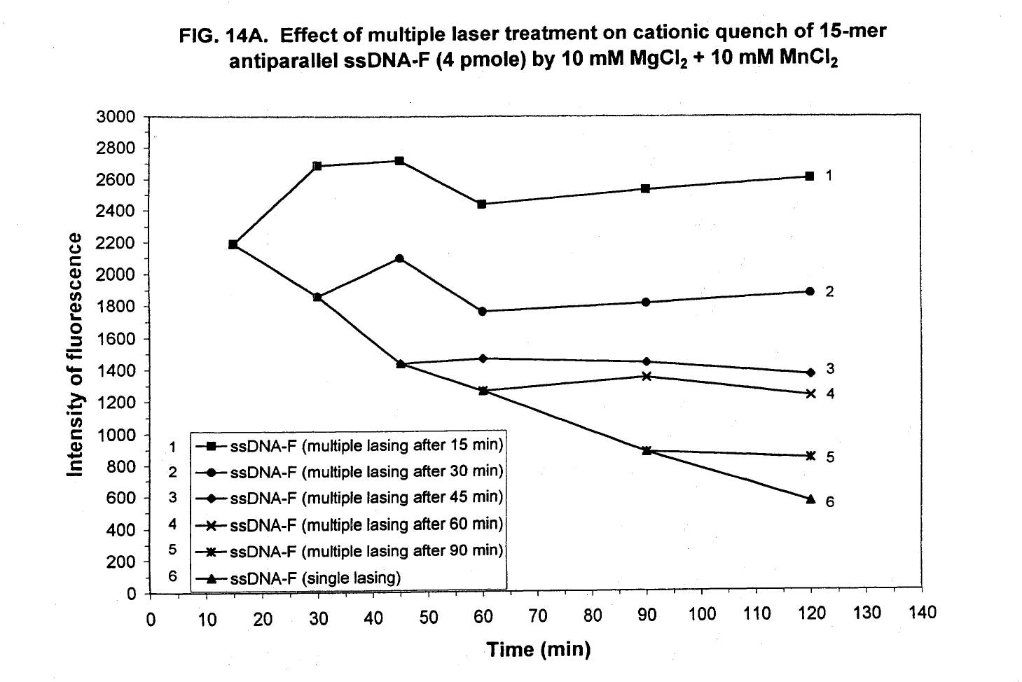

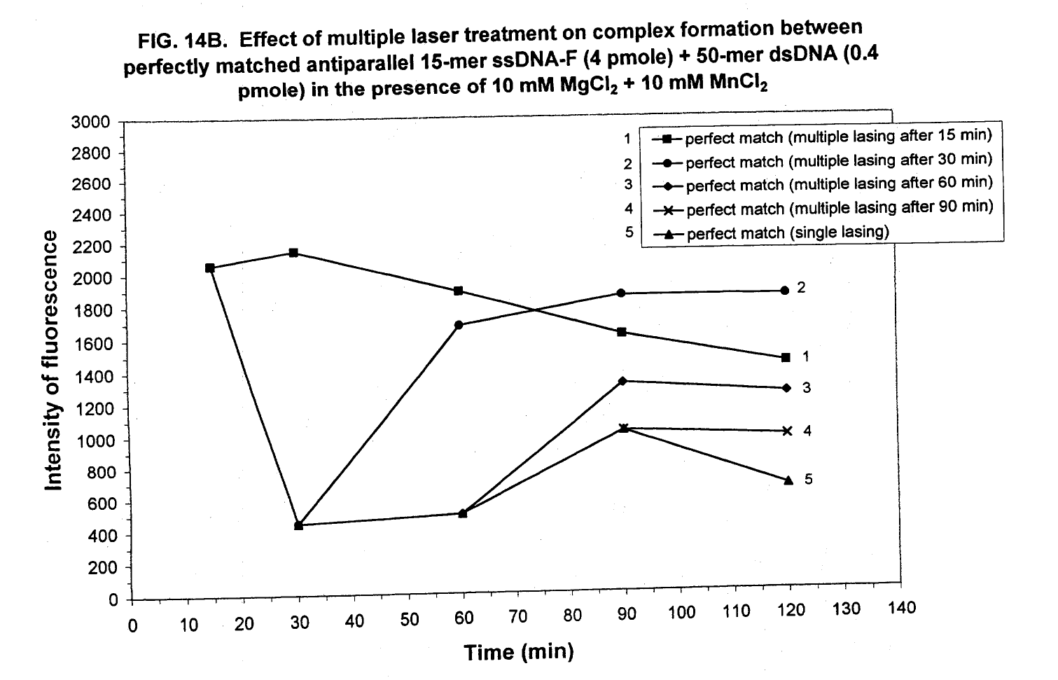

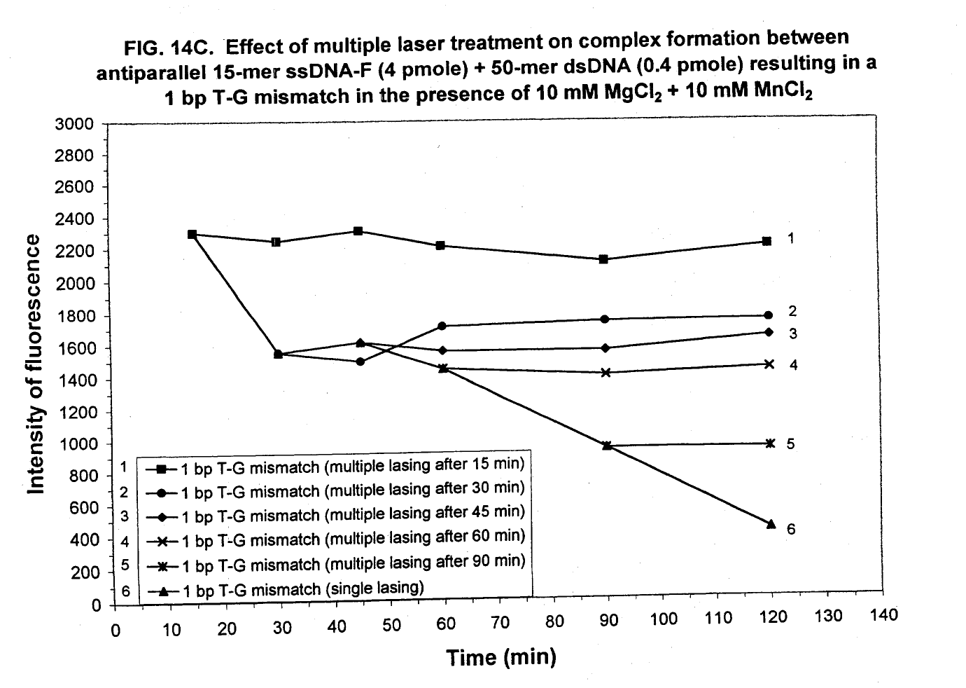

Each hybridization reaction mixture (4 μl) contained the following: 0.4 pmoles of non-denatured target dsDNA, 4 pmoles of 5′-fluorescein labeled ssDNA probe, 10 mM Tris-HCl, pH 7.5, 10 mM MgCl₂ and 10 mM MnCl₂. The reaction mixtures were incubated at room temperature (21° C.) for various periods ranging from 1 minute to 2 hours. Following incubation, samples were placed into a quartz cuvette, irradiated once with an argon ion laser beam having a wavelength of 488 nm and monitored for fluorescent emission. Further fluorescent measurements were taken of the same samples after subsequent multiple laser irradiation, at the indicated times (FIG. 14). The intensity of fluorescence was plotted as a function of time for each sample analyzed.

The fluorescence emitted by control samples comprising 4 pmoles of Probe No. 5 plus 10 mM MgCl₂ and 10 mM MnCl₂, in the absence of target dsDNA, dramatically decreased 3-fold within just 5 minutes of incubation (data not shown), and then steadily declined at a much slower rate within the next few hours (FIG. 14A). This effect we refer to as “Cationic Quench”. This inhibition of fluorescence, associated with increased incubation periods of ssDNA-F probes with specific cations, occurred routinely in the presence of divalent cations, but not in the presence of monovalent cations (data not shown). This observation makes evident the importance of incubating the control sample in an experiment under exactly the same conditions that the test samples of an experiment are reacted. Multiple lasing of each ssDNA-F control sample after varying periods of incubation inhibited further quenching of the fluorophore, resulting in a steady level of fluorescence thereafter (FIG. 14A). This result was entirely unanticipated.

When the antiparallel ssDNA-F Probe No. 5 was incubated with the 50-mer wild-type dsDNA target (SEQ ID NO:5) in the presence of 10 mM MgCl₂ and 10 mM MnCl₂, dsDNA:ssDNA-F complex formation was evident after 15 minutes of incubation resulting in a decrease in fluorescence, which was 6% greater than the progressive cationic quench of the control Probe No. 5 (compare FIGS. 14A and 14B). Complex formation was greatly indicated after 30 and 60 minutes of incubation of SEQ ID NO:5 with Probe No. 5 in the presence of 10 mM MgCl₂ and 10 mM MnCl₂, generating a 76% and 61% decrease in fluorescence, respectively, compared to that achieved by the cationically quenched Probe No. 5 alone (FIG. 14B). After 90 and 120 minutes of incubation in the presence of 10 mM MgCl₂ and 10 mM MnCl₂, no complex formation was being signaled (FIG. 14B). The level of fluorescent emission seen at 90 and 120 minutes was wholly attributable to the cationic quench effect (compare FIGS. 14A and 14B).

By contrast, dsDNA:ssDNA-F complexes that contained a 1 bp T-G mismatch (SEQ ID NO:7+Probe No. 5) formed at a slower rate and were much less stable once formed in the presence of 10 mM MgCl₂ and 10 mM MnCl₂. The 1 bp T-G mismatched complex was first observed after 30 minutes of incubation, and appeared to have been eliminated after 60 minutes of incubation (FIG. 14C). Once again, the probe was antiparallel to the complementary strand in the duplex (FIG. 14C).

Multiple laser irradiation of perfectly complementary dsDNA:ssDNA complexes (SEQ ID NO:5+Probe No. 5) formed after 30 minutes or 60 minutes of incubation in the presence of 10 mM MgCl₂ and 10 mM MnCl₂ resulted in fluorescent emissions consistent with the destruction of these complexes at a rate characteristic for DNA complexes containing an antiparallel ssDNA probe (FIG. 14B). When a subsequent measurement was made at 45 minutes after lasing of the perfectly complementary complex at 30 minutes, the emission intensity level was 1869, testimony to the rapidity with which the complex was destroyed (data not shown). The level of fluorescent emission, after multiple lasing, returned to the cationically quenched values observed by the uncomplexed Probe No. 5 alone control (compare FIGS. 14A and 14B). The only exception was the perfectly matched complexes formed after 15 minutes of incubation and repeatedly irradiated thereafter (FIG. 14B). In this case the fluorescent emission was not consistent with the destruction of the complexes (FIG. 14B), even though further cationic quench of Probe No. 5, when multiply irradiated after a 15 minute incubation, was totally inhibited (FIG. 14A). DsDNA:ssDNA complexes containing a 1 bp T-G mismatch (SEQ TD NO:7+Probe No. 5) were similarly apparently destroyed by multiple lasing (FIG. 14C).

An experiment was performed to determine the basis for the effect of multiple lasing on the complexes. It was found that when fresh cations were added to the reaction mixture which had been lased twice, the inhibition of cationic quench in fluorescence emitted by the ssDNA-F probe could not be reversed and further cationic quench did not occur upon further incubation, strongly suggesting that the ssDNA-F probe was inactivated by multiple irradiation, by a yet unknown mechanism (data not shown). Similarly, when fresh ssDNA-F probes were added to the reaction mixture which had been lased twice, after normalizing for the increased fluorescent emission of the fresh probe, no subsequent progressive cationic quenching was observed upon further incubation of the reaction mixture, strongly suggesting that the lased cations were somehow disabled (data not shown).

In the foregoing examples and description, we have elucidated that heteropolymeric nucleic acid strands can specifically bind on the basis of homologous base pairing. Such binding can occur between parallel or antiparallel strands.

We have also elucidated that nucleic acid bases bound in a Watson-Crick complementary duplex are not quiescent as regards the bases of proximal nucleic acid strands and that such bases can be interacted with on the basis of Watson-Crick complementary base pairing or homologous base pairings, depending on the binding potential of the proximal sequence of bases determined by either of the possible binding motifs. This is true whether the bases in the duplex are interacting with bases in a third strand to form a specifically bound triplex structure or whether the bases of the duplex are specifically interacting with proximal bases which are themselves coupled into a Watson-Crick complementary duplex. Accordingly the invention comprises the discovery that Watson-Crick coupled bases remain reactive as specific bases to interact and bind to proximal bases on other strands and do so with great specificity and alacrity. While all of this is remarkable, it is considered especially remarkable that A:T and G:C pairings are detected as mismatches in binding reactions wherein the homologous binding motif is dominant and being enforced on all base pairs by a strand-wide imperative. It is likewise remarkable that homologous quadruplex binding is more specific than is Watson-Crick complementary quadruplex binding. Quadruplex binding can occur between the major groove side of a duplex-coupled base or base pair and the minor groove side of a duplex-coupled base or base pair. Heretofore, while the potential of further binding by a base already complexed in a duplex was unknown, it had been postulated that third strand recognition of bases in a duplex occurred solely in the major groove of the duplex. This we show is not the case. We have also demonstrated that putative backbone repulsion is no barrier to specific duplex:duplex interaction.

Our invention relates to readily achieved binding reactions which can be achieved with short incubation periods even at room temperature and which do not depend on molar excess of a reagent to drive a reaction. Accordingly the invention is shown to be not only readily achieved, but obviously biologically relevant.

Finally we have shown that partial Watson-Crick complementary conjugation with free bases can contribute to increased duplex binding and increased specificity.

It is most remarkable to detect specific homologous recognition and binding by bases previously and stably coupled into Watson-Crick complementary duplexes.

While the invention has been described in detail and with reference to specific examples thereof, it will be apparent to one skilled in the art that various changes and modifications can be made therein without departing from the spirit and scope thereof.

What is claimed is:

1. A method for modifying transcription and/or translation in an organism, said method comprising:
administering to the organism a composition comprising a probe containing a heteropolymeric probe sequence of nucleic acids or nucleic acid analogues; and
binding the probe to a target to modify transcription and/or translation in the organism, wherein the target is in the organism and contains a heteropolymeric target sequence of nucleic acids,
wherein the heteropolymeric probe sequence is bonded to the heteropolymeric target sequence to form a complex by Watson-Crick complementary base interaction or by homologous base interaction, provided that when the complex is a duplex and the heteropolymeric probe sequence is antiparallel to the heteropolymeric target sequence, the heteropolymeric probe sequence is bonded to the heteropolymeric target sequence by homologous base interaction, and provided that when the complex is a triplex, the complex is free of RecA protein.

2. The method of claim 1, wherein a phenotype and/or a genotype of the organism is modified.

3. The method of claim 1, wherein a disorder of the organism is prevented or treated.

4. The method of claim 3, wherein the complex is a duplex and the heteropolymeric probe sequence is bound to the heteropolymeric target sequence by homologous base interaction or by Watson-Crick complementary base interaction and the heteropolymeric probe sequence is parallel or antiparallel to the heteropolymeric target sequence.

5. The method of claim 3, wherein one of the probe and the target is single-stranded, the other of the probe and the target is double-stranded, and the complex is a triplex.

6. The method of claim 5, wherein the complex is free of PNA.

7. The method of claim 5, wherein the heteropolymeric probe sequence and the heteropolymeric target sequence have parallel directionality, and the heteropolymeric probe sequence is bonded to the heteropolymeric target sequence by homologous base interaction.

8. The method of claim 5, wherein the heteropolymeric probe sequence and the heteropolymeric target sequence have parallel directionality, and the heteropolymeric probe sequence is bonded to the heteropolymeric target sequence by Watson-Crick complementary base interaction.

9. The method of claim 5, wherein: (a) the heteropolymeric probe sequence and the heteropolymeric target sequence have parallel directionality; (b) the target includes a first strand containing the heteropolymeric target sequence and a second strand containing a second heteropolymeric target sequence complementary and antiparallel to the first heteropolymeric target sequence; and (c) the heteropolymeric probe sequence is bonded to the heteropolymeric target sequence by homologous base interaction and the heteropolymeric probe sequence is bonded to the second heteropolymeric target sequence by Watson-Crick complementary base interaction.

10. The method of claim 5, wherein the heteropolymeric probe sequence and the heteropolymeric target sequence have antiparallel directionality, and the heteropolymeric probe sequence is bonded to the heteropolymeric target sequence by homologous base interaction.

11. The method of claim 5, wherein the heteropolymeric probe sequence and the heteropolymeric target sequence have antiparallel directionality, and the heteropolymeric probe sequence is bonded to the heteropolymeric target sequence by Watson-Crick complementary base interaction.

12. The method of claim 5, wherein: (a) the heteropolymeric probe sequence and the heteropolymeric target sequence have antiparallel directionality; (b) the target includes a first strand containing the heteropolymeric target sequence and a second strand containing a second heteropolymeric target sequence complementary and antiparallel to the first heteropolymeric target sequence; and (c) the heteropolymeric probe sequence is bonded to the heteropolymeric target sequence by homologous base interaction and the heteropolymeric probe sequence is bonded to the second heteropolymeric target sequence by Watson-Crick complementary base interaction.

13. The method of claim 3, wherein the probe and the target are double-stranded and the complex is a quadruplex.

14. The method of claim 13, wherein the complex is free of PNA.

15. The method of claim 13, wherein the heteropolymeric probe sequence and the heteropolymeric target sequence have parallel directionality, and the heteropolymeric probe sequence is bonded to the heteropolymeric target sequence by homologous base interaction.

16. The method of claim 13, wherein the heteropolymeric probe sequence and the heteropolymeric target sequence have parallel directionality, and the heteropolymeric probe sequence is bonded to the heteropolymeric target sequence by Watson-Crick complementary base interaction.

17. The method of claim 13, wherein: (a) the heteropolymeric probe sequence and the heteropolymeric target sequence have parallel directionality; (b) the probe includes a first probe strand containing the heteropolymeric probe sequence and a second probe strand containing a second heteropolymeric probe sequence complementary and antiparallel to the first heteropolymeric probe sequence; (c) the target includes a first target strand containing the heteropolymeric target sequence and a second target strand containing a second heteropolymeric target sequence complementary and antiparallel to the first heteropolymeric target sequence; and (d) the heteropolymeric probe sequence is bonded to the heteropolymeric target sequence by homologous base interaction and the heteropolymeric probe sequence is optionally bonded to the second heteropolymeric target sequence by Watson-Crick complementary base interaction.

18. The method of claim 13, wherein: (a) the heteropolymeric probe sequence and the heteropolymeric target sequence have parallel directionality; (b) the probe includes a first probe strand containing the heteropolymeric probe sequence and a second probe strand containing a second heteropolymeric probe sequence complementary and antiparallel to the first heteropolymeric probe sequence; (c) the target includes a first target strand containing the heteropolymeric target sequence and a second target strand containing a second heteropolymeric target sequence complementary and antiparallel to the first heteropolymeric target sequence; and (d) the heteropolymeric probe sequence is bonded to the heteropolymeric target sequence by Watson-Crick complementary base interaction and the heteropolymeric probe sequence is optionally bonded to the second heteropolymeric target sequence by homologous base interaction.

19. The method of claim 13, wherein: (a) the heteropolymeric probe sequence and the heteropolymeric target sequence have antiparallel directionality; (b) the probe includes a first probe strand containing the heteropolymeric probe sequence and a second probe strand containing a second heteropolymeric probe sequence complementary and antiparallel to the first heteropolymeric probe sequence; (c) the target includes a first target strand containing the heteropolymeric target sequence and a second target strand containing a second heteropolymeric target sequence complementary and antiparallel to the first heteropolymeric target sequence; and (d) the heteropolymeric probe sequence is bonded to the heteropolymeric target sequence by homologous base interaction and the heteropolymeric probe sequence is optionally bonded to the second heteropolymeric target sequence by Watson-Crick complementary base interaction.

20. The method of claim 13, wherein: (a) the heteropolymeric probe sequence and the heteropolymeric target sequence have antiparallel directionality; (b) the probe includes a first probe strand containing the heteropolymeric probe sequence and a second probe strand containing a second heteropolymeric probe sequence complementary and antiparallel to the first heteropolymeric probe sequence; (c) the target includes a first target strand containing the heteropolymeric target sequence and a second target strand containing a second heteropolymeric target sequence complementary and antiparallel to the first heteropolymeric target sequence; and (d) the heteropolymeric probe sequence is bonded to the heteropolymeric target sequence by Watson-Crick complementary base interaction and the heteropolymeric probe sequence is optionally bonded to the second heteropolymeric target sequence by homologous base interaction.

21. The method of claim 3, wherein the probe occupies a minor groove of the target.

22. The method of claim 3, wherein the probe occupies a major groove of the target.

23. The method of claim 5, wherein bases of the probe interact with bases of both strands of the target simultaneously.

24. The method of claim 13, wherein bases of the probe interact with bases of both strands of the target simultaneously.

25. The method of claim 3, wherein a backbone of the probe comprises deoxyribose phosphate.

26. The method of claim 3, wherein a backbone of the probe is uncharged, partially negatively charged, or positively charged.

27. The method of claim 3, wherein at least one base in the probe is a radical cation.

28. The method of claim 3, wherein the probe further comprises at least one terminal blocking agent effective to hinder digestion of the probe.

29. The method of claim 3, wherein said probe further comprises at least one binding promoter.

30. The method of claim 29, wherein said at least one binding promoter is a condensing agent or a decondensing agent.

31. The method of claim 29, wherein said at least one binding promoter is a member selected from the group consisting of YOYO-1, TOTO-1, YOYO-3, TOTO-3, POPO-1, BOBO-1, POPO-3, BOBO-3, LOLO-1, JOJO-l, cyanine dimers, YO-PRO-1, TO-PRO-1, YO-PRO-3, TO-PRO-3, TO-PRO-5, PO-PRO-1, BO-PRO-1, PO-PRO-3, BO-PRO-3, LO-PRO-1, JO-PRO-1, cyanine monomers, ethidium bromide, ethidium homodimer-1, ethidium homodimer-2, ethidium derivatives, acridine, acridine orange, acridine derivatives, ethidium-acridine heterodimer, ethidium monoazide, propidium iodide, SYTO dyes, SYBR Green 1, SYBR dyes, Pico Green, SYTOX dyes and 7-aminoactinomycin D.

32. The method of claim 29, wherein a concentration of the at least one binding promoter is provided to favor one binding structure of the complex over other possible binding structures of the complex.

33. The method of claim 3, further comprising at least one free base, nucleotide, nucleoside, cationic polypeptide, monovalent cation or transition metal cation bonded to at least one of the probe and the target.

34. The method of claim 3, wherein the probe is heteropolymeric PNA bonded to the heteropolymeric target sequence by homologous base interaction, and the target is a duplex.

35. The method of claim 3, wherein the probe is a duplex and at least one strand of the probe is uncharged, partially negatively charged or positively charged, and the target is single-stranded or double-stranded.

36. The method of claim 3, wherein the probe comprises a member selected from the group consisting of chemotherapeutic agents, nucleic acid cleaving agents, nucleases, polymerases, transcription factors, crosslinking agents, intercalators, minor groove binders, photoactive agents, labels, duplex binding agents, triplex binding agents, quadruplex binding agents, multimeric binding agents, proteins, peptides, recombinases, spacers and linkers.

37. The method of claim 3, wherein the probe is provided as a product of transcription, expression or digestion in a cell or an organism.

38. The method of claim 3, wherein the probe comprises a vector, a transfer vehicle, a transfection vehicle or a cell-uptake component.

39. The method of claim 3, wherein the target comprises nucleic acids that are methylated, telomeric or in an A, B or Z conformation.

40. The method of claim 3, wherein the target is genomic DNA.

41. The method of claim 3, wherein the target comprises a promoter sequence, a coding sequence, a promoter sequence and an adjacent coding sequence, a non-coding sequence or a repetitive sequence.

42. The method of claim 3, wherein the target comprises mitochondrial DNA or mitochondrial RNA.

43. The method of claim 3, wherein the target comprises viral, fungal or bacterial nucleic acids.

44. The method of claim 3, wherein the target comprises mRNA, hRNA, mtRNA, rRNA, tRNA, rDNA or snRNA.

45. The method of claim 3, wherein a gene associated with the target is completely or partially transcriptionally silenced.

46. The method of claim 45, wherein expression of the gene is modulated by adjusting a binding affinity of the probe for the target.

47. The method of claim 3, wherein the method causes cell apoptosis or necrosis.

48. The method of claim 3, further comprising forming a transgenic organism.

49. The method of claim 3, wherein the probe is introduced into a bacterium and the bacterium is then internalized by the organism.

50. The method of claim 1, wherein a gene associated with the transcription and/or the translation is identified.

51. The method of claim 1, wherein a phenotype of the organism is affected.

52. The method of claim 3, wherein heterochromatin is formed.

53. A method for preventing or treating a disorder in an organism, said method comprising:
administering to the organism a composition comprising a probe containing a heteropolymeric probe sequence of nucleic acids or nucleic acid analogues; and
binding the probe to a target to prevent or treat the disorder in the organism, wherein the target is in the organism and contains a heteropolymeric target sequence of nucleic acids,
wherein the heteropolymeric probe sequence is bonded to the heteropolymeric target sequence to form a complex by Watson-Crick complementary base interaction or by homologous base interaction, provided that when the complex is a duplex and the heteropolymeric probe sequence is antiparallel to the heteropolymeric target sequence, the heteropolymeric probe sequence is bonded to the heteropolymeric target sequence by homologous base interaction, and provided that when the complex is a triplex, the complex is free of RecA protein.
